(19) United States
(12) Patent Application Publication
(10) Pub. No.: US 2007/0149483 A1
(43) Pub. Date: Jun. 28, 2007

(54) Methods and compounds for modulating triglyceride and vldl secretion

(76) Inventors: James C. Jamieson, Manitoba (CA); Gro Thorne-Tjomsland, Manitoba (CA); Zemin Yao, Ontario (CA); Andrea Marat, Montreal (CA); Khai Tran, Ontario (CA)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 10/582,288

(22) PCT Filed: Dec. 13, 2004
(86) PCT No.: PCT/CA04/02119

§ 371 Date: Dec. 14, 2006

Related U.S. Application Data
(60) Provisional application No. 60/528,723, filed on Dec. 12, 2003.

(51) Int. Cl. A61K 31/685; G01N 33/567

(52) U.S. Cl. 514/78; 435/7.2

(57) ABSTRACT

The invention provides uses of autophagocytosis inducing compounds for reducing serum levels of triglycerides and VLDL and the preparation of medicaments. The invention also provides the use of autophagocytosis inducing compounds for treating hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, or diabetes, insulin resistance, hemodialysis, glycogen storage disease type I, polycystic ovary syndrome, combination thereof. The invention further provides methods of identifying compounds which modulate autophagocytosis.

### FIELD OF INVENTION

The present invention relates to methods and compounds for modulating triglyceride and VLDL secretion.

### BACKGROUND OF THE INVENTION

Hypertriglyeridemia has been identified as a risk factor for cardiovascular disease. Hypertriglyceridemia is generally defined as fasting levels of triglycerides (TG) greater than 200 mg/dL. Elevations in serum levels of TG may result from either increased TG secretion or decreased TG degradation.

The liver secretes TG in the form of very low density lipoprotein (VLDL) that are heterogeneous in size and metabolic fate (Packard and Shepherd, 1997, Arterioscler. Thromb. Vasc. Biol. 17, 3542-3556). Each VLDL particle contains one copy of apolipoprotein (apo) B100 and various amount of TG (Fisher and Ginsberg, 2002, J. Biol. Chem. 277, 17377-17380). In rat hepatoma McA-RH7777 cells, assembly of VLDL is accomplished post-translationally in a post-endoplasmic reticulum (ER) compartment (Tran et al., 2002, J. Biol. Chem. 277, 31187-31200). After its synthesis, apoB100 exits the ER and traverses the cis/medial Golgi in a membrane-associated form associated with little lipids; complete assembly of bulk TG with apoB100 to form VLDL does not occur until apoB100 reaches the distal Golgi (Tran et al., 2002). Formation of the lipid-poor primordial lipoprotein particles in the ER is referred as first-step assembly, whereas incorporation of bulk TG into VLDL within post-ER compartments is known as second-step assembly (Rustaeus et al., 1999, J. Nutr. 129, 463S-466S; Stillemark et al., 2000, J. Biol. Chem. 275, 10506-10513). Factors affecting first-step assembly often govern folding of the nascent apoB100 polypeptide chain, either through post-translational modification (e.g. disulfide bond formation (Tran et al., 1998, J. Biol. Chem 273, 7244-7251) or N-linked glycosylation (Vukmirica et al., 2002, J. Lipid Res. 43, 1496-1507)) or through the interaction of apoB100 with microsomal triglyceride transfer protein (MTP) (Dashti et al., 2002, Biochemistry 41, 6978-6987). Recently, a point mutation R463W associated with familial hypobetalipoproteinemia was identified within the MTP-binding region of apoB that causes impaired first-step assembly (Burnett et al., 2003, J. Biol. Chem. 278, 13442-13452). Features associated with attenuated first-step assembly include enhanced intracellular degradation of newly synthesized apoB100 and decreased secretion of apoB100 proteins. Degradation of misfolded nascent apoB100 in the ER is usually mediated by the ubiquitin-proteosomal system (Fisher and Ginsberg, 2002; Yao et al., 1997 J. Lipid Res 38, 1937-1953).

On the other hand, factors affecting second-step assembly are generally of a lipid nature. Increasing experimental evidence suggests that phospholipid composition of membranes along the secretory pathway is an important determinant of second-step assembly. Previous studies using agents that perturb membrane phospholipid composition by directly (Asp et al., 2000, J. Biol. Chem. 275, 26285-26292; Nishimaki-Mogami et al., 2002, J. Lipid Res. 43, 1035-1045; Tran et al., 2000, J. Biol. Chem 275, 25023-25030) or indirectly (McLeod et al., 1996, J. Biol. Chem. 271, 18445-18455; Wang et al., 1999, J. Biol. Chem. 274, 27793-27800; Yao and Vance, 1988, J. Biol. Chem. 263, 2998-3004) altering the activity of phospholipid-modifying enzymes have identified several such factors. Among them are phosphatidylcholine (PC) and phosphatidylethanolamine (PE) species enriched with oleoyl (18:1(n-9)) chains that create a microsomal membrane milieu permissive to VLDL assembly (Tran et al., 2000). Formation of 18:1(n-9)-rich phospholipid species can be achieved through phospholipid remodelling (i.e., deacylation and reacylation) mediated in part by calcium-independent phospholipase A₂ (iPLA₂) in liver cells (Tran et al., 2000). Turnover of these phospholipids also donates 18:1(n-9) acyl chain for TG synthesis (Tran et al., 2000) and for formation of signaling molecules such as 18:1(n-9)-phosphatidic acid and 18:1(n-9)-diglyceride that play a key role in membrane movement and fusion (Antonny et al., 1997, J. Biol. Chem. 272, 30848-30851; Chemomordik et al., 1995, J. Membr. Biol. 146, 1-14). Limiting incorporation of 18:1(n-9) into membrane phospholipid by oleate deprivation (McLeod et al., 1996), reducing phospholipid remodelling by iPLA₂ inhibition (Tran et al., 2000), and decreasing formation of phosphatidic acid by inhibition of ADP-ribosylation factor-dependent phospholipase (D Asp et al., 2000) in McA-RH7777 cells invariably result in reduced VLDL assembly at the second step. The hallmark of impaired second-step assembly is the secretion of dense, TG-poor apoB100-containing lipoproteins (LpBs). Secretion-incompetent LpBs are destined for degradation by a yet unknown mechanism. A non-proteosomal and post-ER degradation mechanism has been postulated to eliminate abnormal LpBs formed after apoB exits the ER (i.e., in second-step assembly) under various conditions (Fisher et al., 2001, J. Biol. Chem. 276, 27855-27863; Phung et al., 1997, J. Biol. Chem. 272, 30693-30702; Wang et al., 1995, J. Biol. Chem. 270, 24924-24931).

The present inventors have now determined that alterations to membrane phospholipid composition and remodelling inhibit second-step VLDL assembly and activate post-ER degradation.

### SUMMARY OF THE INVENTION

The present inventors have now determined that alterations to membrane phospholipid composition and remodelling inhibit second-step VLDL assembly and activate post-ER degradation.

The invention teaches a method of reducing serum levels of triglycerides and/or VLDL comprising administering a therapeutically effective amount of an autophagocytosis inducing compound to a patient in need thereof.

The invention teaches a use of an autophagocytosis inducing compound for preparing a medicament useful for reducing serum levels of triglycerides and/or cholesterol.

The invention teaches a method of treating or preventing a disorder selected from a group consisting of: hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia or combination thereof comprising administering a therapeutically effective amount of an autophagocytosis inducing compound to a patient in need thereof.

The invention teaches a use of an autophagocytosis inducing compound for the preparation of a medicament useful for treating or preventing a disorder selected from a group consisting of: hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia, or a combination thereof.

In an embodiment of the invention, the autophagocytosis inducing compound may be Map1LC3, GABARAP, GATE16, or Class III P13′kinase.

The invention teaches a method of identifying autophagocytosis modulating compounds comprising: (a) providing a control cell culture system and a test cell culture system; (b) administering a test compound to cells in said test cell culture system; and (c) assaying for autophagocytosis markers in said control cell culture system and said test cell culture system; wherein an abnormal value for said autophagocytosis markers in said test cell culture system as compared to said control cell culture system indicates that the test compound modulates autophagocytosis.

In an embodiment of the invention, the autophagocytosis markers are VLDL and VLDL precursors in ER and Golgi cell fractions.

In another embodiment of the invention, the VLDL precursors are PC moiety containing lipids. The PC moiety containing lipid may be 18:1(n-9) PC.

In a further embodiment of the invention, the VLDL precursors are PE moiety containing lipids. The PE moiety containing lipid may be 20:5(n-3) PE.

In a still further embodiment of the invention, the autophagocytosis markers are determined by detecting the degree of co-localization of apoB100 and Map1LC3 by immunofluorescence.

The invention teaches a method of identifying autophagocytosis inducing compounds comprising: (a) providing a control cell culture system and a test cell culture system; (b) administering a test compound to cells in said test cell culture system; and (c) assaying for autophagocytosis markers in said control cell culture system and said test cell culture system; wherein an abnormal value for said autophagocytosis markers in said test cell culture system as compared to said control cell culture system indicates that the test compound modulates autophagocytosis.

In an embodiment of the invention, the autophagocytosis marker is a PC moiety containing lipid. The PC moiety containing lipid may be 18:1(n-9) PC.

In a further embodiment of the invention, the autophagocytosis marker is a PE moiety containing lipid. The PE moiety containing lipid may be 20:5(n-3) PE.

In an embodiment of any of the methods of the invention, the cells are hepatocytes or hepatoma cells. The cells may be rat hepatocytes which express human apoB100 or rat hepatoma cells which express human apoB100. The rat hepatoma cells may be McA-RH-7777 cells. The apoB100 may be fused with a tag such as fluorescent protein or tetra-cysteine.

The invention teaches a use of an autophagocytosis inducing compound identified by a method of according to the invention, for preparing a medicament useful for reducing serum levels of triglycerides and/or VLDLs.

The invention teaches a pharmaceutical composition comprising an autophagocytosis inducing compound identified by a method according to the invention and a pharmaceutically acceptable carrier.

The invention teaches a method of treating or preventing a disorder selected from a group consisting of: hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia, or combination thereof comprising administering a therapeutically effective amount of the pharmaceutical composition comprising an autophagocytosis inducing compound identified by a method according to the invention and a pharmaceutically acceptable carrier.

### BRIEF DESCRIPTION OF THE FIGURES

FIG. 1A illustrates the density distribution of apoB100 associated with lipoproteins found in conditioned medium for oleate and EPA treated cells. The top panel consists of representative fluorograms. The bottom panel is a line graph illustrating the distribution of [³⁵S]apoB100 associated with each fraction.

FIG. 1B illustrates the density distribution of apoB100 associated with lipoproteins found in the lumenal content of microsomes obtained from oleate and EPA treated cells. The top panel consists of representative fluorograms. The bottom panel is a line graph illustrating the distribution of [³⁵S]apoB100 associated with each fraction.

FIG. 2A comprises line graphs illustrating the pulse-chase analysis for apoB100 from total cell lysates of oleate and EPA treated cells. The top graph expresses the data as the absolute amount of radioactivity associated with [³⁵S]apoB100 at the end of the 1 hour pulse. The bottom graph expresses the data as percent of the initial counts associated with [³⁵S]apoB100 at the end of the 1 hour pulse.

FIG. 2B comprises line graphs illustrating the pulse-chase analysis for apoB100 from conditioned medium from oleate and EPA treated cells. The top graph expresses the data as the absolute amount of radioactivity associated with [³⁵S]apoB100 at the end of the 1 hour pulse. The bottom graph expresses the data as percent of the initial counts associated with [³⁵S]apoB100 at the end of the 1 hour pulse.

FIG. 2C comprises line graphs illustrating the pulse-chase analysis for apoA-1 from total cell lysates of oleate and EPA treated cells. The top graph expresses the data as the absolute amount of radioactivity associated with [³⁵S]apoA-1 at the end of the 1 hour pulse. The bottom graph expresses the data as percent of the initial counts associated with [³⁵S]apoA1 at the end of the 1 hour pulse.

FIG. 2D line graphs illustrating the pulse-chase analysis for apoA-1 from conditioned medium from oleate and EPA treated cells. The top graph expresses the data as the absolute amount of radioactivity associated with [³⁵S]apoA-1 at the end of the 1 hour pulse. The bottom graph expresses the data as percent of the initial counts associated with [³⁵S]apoB100 at the end of the 1 hour pulse.

FIG. 3A comprises line graphs comparing membrane associated apoB100 trafficking in the ER (top panel), cis/medial Golgi (middle panel) and distal Golgi (bottom panel) for oleate and EPA treated cells, at the end of 20 min pulse.

FIG. 3B illustrates the results of immunopreciptation and SDS-PAGE/fluorography analysis of apoB100 for lumenal fractions of ER, cis/medial Golgi, and distal Golgi from oleate and EPA treated cells after 20 min pulse and 45 min chase.

FIG. 4A is a bar graph illustrating the depicting the diameters of pooled particles within Golgi saccules 1-3 (cis-Golgi).

FIG. 4B is a bar graph illustrating the depicting the diameters of pooled particles within Golgi saccules 4-6 (trans-Golgi)+TGN.

FIG. 4C illustrates the particle size range for Types I-V particles.

FIGS. 5A, 5B, 5C, 5D and 5E are transmission electron microscope images of five types of lipid/lipoprotein particles identified in the Golgi and associated vacuoles.

FIGS. 6A, 6B, 6C, 6D and 6E are transmission electron microscope images showing the formation of lipid/lipoprotein-containing vacuoles in the trans-Golgi region of EPA treated cells.

FIG. 7 illustrates the results of immunofluorescent microscopy analysis of untreated, oleate treated and EPA treated cells blotted with anti-human apoB100 antibody and anti-rat Map1LC3 antibody. The arrowheads in the merge images illustrate the co-localization of apoB100 and Map1LC3.

FIG. 8 illustrates the results of immunofluorescent microscopy analysis of untreated, oleate treated and EPA treated cells labelled with monodansylcadaverine.

FIG. 9A comprises line graphs illustrating the distribution of [¹⁴C]oleate (top panel) and [³H]EPA (bottom panel) in PC, PE, and TG lipids for cell lysates from oleate and EPA treated cells.

FIG. 9B comprises line graphs illustrating the secretion of [¹⁴C]oleate (top panel) and [³H]EPA (bottom panel) labelled TG and FFA lipids for oleate and EPA treated cells.

FIG. 10A comprises bar graphs illustrating the distribution of [¹⁴C]oleate labelled PC, PE, and TG between cytosol (top panel), microsomal membranes (middle panel) and microsomal lumen (bottom panel).

FIG. 10B comprises bar graphs illustrating the distribution of [³H]EPA labelled PC, PE, and TG between cytosol (top panel), microsomal membranes (middle panel) and microsomal lumen (bottom panel).

FIG. 10C comprises line graphs illustrating the incorporation of [¹⁴C]oleate and [³H]EPA into PC (top) and PE (bottom).

FIG. 11 is a diagrammatic representation of the relationship between phospholipid remodelling/turnover and the distribution of metabolically distinct TG pools.

### DETAILED DESCRIPTION

Activation of Post-ER Degradation Decreases TG and VLDL Secretion

While the invention is not limited to any particular mechanism, it is believed that TG and VLDL secretion can be modulated by promoting post-ER degradation of lipid/lipoproteins by inducing autophagocytosis. The inventors have determined that alterations to membrane phospholipid composition and remodelling inhibit second-step VLDL assembly. In particular, the inventors have determined that alterations in membrane phosphotidylcholine (PC) to phopsphatidylethanolamine (PE) ratio are associated with intracellular accumulation of triglycerides and the activation of post-ER degradation.

The inhibitory effect on TG secretion in vitro (Lang and Davis, 1990, J. Lipid Res. 31, 2079-2086; Wong and Nestel, 1987, Atherosclerosis 64, 139-146) and the plasma TG-lowering effect of eicosapentaenoic acid (EPA) in vivo (Harris, 1999, Lipids 34 Suppl, S257-S258) have been documented. However, the mechanism of the hypotriglyceridemic effect of EPA has not been clearly elucidated and remains controversial.

The inventors investigated the impact of membrane phospholipid remodelling on second-step VLDL assembly by comparing the effects of oleate with EPA. The inventors hypothesized that incorporation of 20:5(n-3) into phospholipid and subsequently into TG through remodelling creates a lipid environment unfavorable for second-step VLDL assembly. To test this hypothesis, McA-RH7777 cells expressing human apoB100 were cultured under conditions where synthesis and ER exit of apoB100 were unaffected by the EPA treatment. The inventors found that alteration in phospholipid molecular species by exogenous fatty acids appeared to affect the recruitment of TG, which is modulated by its synthesis and intracellular distribution, during second-step VLDL assembly, and to coincide with formation of post-ER degradative compartment.

The inventors found that the second-step assembly of VLDL is regulated by membrane phospholipid remodelling (i.e, deacylation/reacylation) under the influx of exogenous fatty acids. One of the important functional aspects of phospholipid remodelling in relation to VLDL assembly is the utilization of released acyl chain (upon deacylation) in the synthesis of TG. The preferential incorporation of oleate into membrane PC is believed to be mediated by both the de novo and remodelling pathways, for its presence in both sn-1 and sn-2 position of the glycero-backbone of PC. In contrast, the preferential incorporation of EPA into the sn-2 position of membrane phospholipids and it's subsequent transfer from PC to PE are clear indicators of the remodelling process. The intrinsic nature of polyunsaturated fatty acid incorporation into phospholipids through deacylation/reacylation process mediated by intracellular Ca²⁺-independent phospholipase A₂ and PE being the preferential destination pool for EPA incorporation have recently demonstrated in other cell types (Balsinde, 2002). Upon influx of exogenous fatty acids, both oleate and EPA released from phospholipid remodelling are utilized for TG synthesis with little selectivity. However, the inventors found that 20:5-containing TG was poorly secreted as compared with 18:1-containing TG, suggesting that 20:5-TG is inefficiently utilized for VLDL assembly. The inventors believe that the intrinsic nature of membrane phospholipid deacylation/reacylation and the differential incorporation of oleate and EPA into PC and PE lead to the formation of different TG pools that may or may not be accessible and efficiently utilized in the second-step assembly.

The inventors have determined that the alteration of membrane PC-to-PE ratio is associated with an accumulation of TG in the cytosolic pool and activation of post-ER degradation. In addition to the importance of PC and PE remodelling in the formation of different TG species (i.e., 18:1-TG versus 20:5-TG), the inventors found that a decrease in the PC-to-PE ratio within the microsomal membrane is associated with impaired second-step VLDL assembly and accumulation of TG in the cytosolic pool. Alteration of PC-to-PE ratio could be attained by changing of either PC or PE content in the microsomal membranes and may be an indicator for the efficiency of the second-step VLDL assembly. The inventors believe that oleate treatment of McA-RH7777 cells increased PC content in the microsomal membranes (Wang et al., 1999), particularly in the ER and distal Golgi. In contrast, EPA treatment resulted in an increase in PE content (thus lowering PC-to-PE ratio) in the membrane of distal Golgi that was effectively preventing VLDL assembly. An increase in liver PE levels has also been reported in EPA-fed rats (Kotkat et al., 1999, Comp Biochem. Physiol A Mol. Integr. Physiol 122, 283-289). Lowering PC-to-PE ratio of liver microsomal membranes that is associated with impaired second-step VLDL assembly (decreased VLDL secretion but not HDL secretion) has been observed in other models such as choline deficiency (Ridgway et al., 1989) and inhibition of PE methylation pathway (Nishimaki-Mogami et al., 2002); (Noga et al., 2002, J. Biol. Chem. 277, 42358-42365). Disruption of PE to PC conversion via the PE methylation pathway by chemical inhibition (Nishimaki-Mogami et al., 2002) or by genetic disruption of PE methyltransferase in mice (Noga et al., 2002) showed reduction of PC-to-PE ratio that was associated with impaired apoB100-VLDL secretion. In PE methyltransferase deficient animals, particularly in males, the increased in liver PE was associated with liver TG accumulation and decreased plasma TG. Unlike primary rat hepatocytes, McA-RH7777 cells lack PE methyltransferase activity (Cui et al., 1995, Biochem. J. 312, 939-945) and are unable to assemble VLDL unless exogenous oleate is supplemented to the medium. The restoration of VLDL assembly in McA-RH7777 cells in the presence of exogenous oleate may in part be resulted from re-establishing of PC-to-PE ratio (due to elevation of PC content) permissive for VLDL assembly. Reconstitution of PE methyltransferase activity in McA-RH7777 cells increased secretion of TG in apoB100-VLDL (DeLong et al., 1999) and generated diverse PC species which resembled those synthesized by the methylation pathway in hepatocytes (Noga et al., 2002). The asymmetric distribution of membrane phospholipids (Daleke, 2003, J. Lipid Res. 44, 233-242) (i.e, PC enriched in the lumenal leaflet and PE enriched in the cytosolic leaflet of the microsomal membranes, particularly at the site of VLDL assembly, the Golgi) together with their intrinsic property of accepting and donating different fatty acyl chains during remodelling, contribute to the formation of two metabolically distinct TG pools. As a result, TG formed in EPA treatment was accumulated more in the cytosolic pool that might be inaccessible for VLDL assembly. It appears that phospholipid remodelling together with the alteration of PC-to-PE ratio induced by different fatty acid treatments have strong impact on TG synthesis/distribution and VLDL assembly.

The inventors investigated the effect of altered PC-to-PE ratio in the membrane of distal Golgi with respect to post-ER degradation. One of the essential proteins involved in the entire process of autophagosome formation is Map1LC3, which exists in two forms: an 18 kDa cytosolic form and a 16 kDa autophagosome membrane-associated form (Kabeya et al., 2000). The yeast homolog Apg8/Aut7p is conjugated to PE when binding to the autophagosome membrane; hence, the membrane-bound Map1LC3 has been postulated as a PE-conjugated form (Ichimura et al., 2000, Nature 408, 488-492). Autophagosome formation begins with formation of a membrane structure termed an “isolation membranes”, postulated to be derived from the ER (Ueno et al., 1991, J. Biol. Chem, 266, 18995-18999), the trans-Golgi network (Yamamoto et al., 1990, J. Histochem. Cytochem. 38, 573-580), and/or a unique, uncharacterized intracellular compartment (Stromhaug et al., 1998, Biochem. J. 335, 217-224), that progressively enwraps the cargo. Fusion between the isolation membrane and the vacuolar membrane leads to formation of autophagosome, which in turn fuses with lysosomes (Yamamoto et al., 1990) to form autophagolysosomes, resulting in degradation of the lumenal contents. The detection by TEM of lipid/lipoprotein-containing vacuoles encased in a double membrane structure near the trans-Golgi, and the increased punctate staining of the autophagocytic markers Map1LC3 and MDC by confocal and fluorescent microscopy, respectively, clearly indicate that autophagy is induced by EPA treatment.

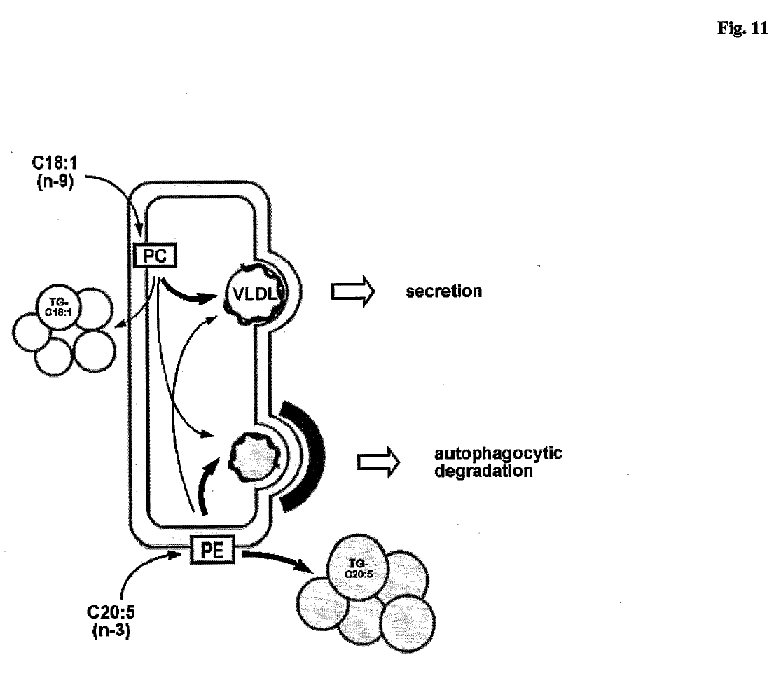

Although the constitutive nature of autophagosome formation is essential for cell survival (Klionsky and Emr, 2000, Science 290, 1717-1721), as it was also detected in both oleate-treated and control cells, the increased autophagy in EPA treatment may play a role in the disposal of accumulated aberrant lipid/lipoproteins in the distal Golgi and/or lipid particles in the cytosol as a result of impairment of second-step assembly. Autophagosome formation in cultured cells can be stimulated by starvation condition (Klionsky and Emr, 2000) or inhibited by wortmannin or 3-methyladenine, inhibitors of phosphatidylinositide 3-kinase (Mizushima et al., 2001, J. Cell Biol. 152, 657-668). In light of the evidence that the non-proteosomal degradation of apoB is sensitive to phosphatidylinositide 3-kinase inhibition (Fisher et al., 2001; Phung et al., 1997), the inventors believe that autophagy represents a missing link for post-ER degradation in VLDL assembly. Thus, while apoB degradation during first-step assembly is known to be mediated by the ubiquitin-proteasome pathway (Fisher and Ginsberg, 2002; Yao et al., 1997), the inventors propose that aberrant lipid/lipoproteins generated from impaired second-step assembly are removed at least in part by autophagy. The relationship between phospholipid remodelling and distribution of metabolically distinct TG pools as well as the autophagosome formation is depicted in FIG. 11.

The inventors have determined that membrane lipids containing 18:1(n-9) and 20:5(n-3) acyl chain in are important in VLDL assembly. Although compartmentalized 18:1(n-9)-TG and 20:5(n-3)-TG pools may explain the difference in how oleate- and EPA-treatment affect second-step assembly, it is also possible that alterations in membrane phospholipid species directly impact VLDL assembly. The molecular species analysis clearly shows that EPA treatment results in marked reduction of membrane-associated PC and PE species containing 18:1(n-9) and in an increase of species containing 20:5(n-3). The inventors have demonstrated previously that in McA-RH7777 cells, reduction of 18:1(n-9) acyl chain in membrane PC and PE, either by oleate deprivation (McLeod et al., 1996) or by inhibition of iPLA₂ (Tran et al., 2000), is closely associated with impaired second-step VLDL assembly. Both studies suggest that oleate does not merely serve as a substrate for the TG synthesis, which precedes or coincides with VLDL assembly. Rather, incorporation of 18:1(n-9) acyl chain into microsomal phospholipids may establish a membrane platform for efficient bulk incorporation of TG into VLDL. Establishing a membrane milieu compatible with second-step assembly is important, especially in view of a large body of evidence that membrane-associated apoB100 within microsomes is the precursor of assembled/secreted VLDL (Tran et al., 2002; Stillemark et al., 2000; Hebbachi and Gibbons, 2001, J. Lipid Res. 42, 1609-1617; Rustaeus et al., 1998, J. Biol. Chem 273, 5196-5203). In this context, the presence of other 18:1(n-9)-containing lipids such as phosphatidic acid and diglyceride which are important for membrane dynamics (Antonny et al., 1997; Chemomordik et al., 1995) may also facilitate the second-step assembly process.

The inventors observed massive accumulation of PE in the Golgi apparatus accompanied with markedly depleted 18:1(n-9)-containing PC in EPA-treated cells. These results reveal for the first time the assembly intermediates of lipid donors and acceptors at the VLDL assembly site. TEM morphometric analysis data of EPA treated cells showed different types of lipid/lipoprotein particles, at the distal Golgi and vacuolar structures, resembling of original lipid donors (Type I), intermediate lipid donors (Types II and III) and nascent lipoproteins (Types IV and V). As membrane associated apoB100 being precursors of VLDL, the impaired second-step assembly was clearly manifested by accumulation of apoB100 in the membrane of distal Golgi and the formation of degradation vacuoles housing intermediate lipid/lipoprotein particles. The tipping towards one side or the other of the balance between post-ER degradation and second-step VLDL assembly can be influenced by alteration of membrane phospholipid species.

Thus, the inventors have identified and characterized an intracellular compartment where post-endoplasmic reticulum degradation of apolipoprotein B and lipid and lipoprotein particles occurs. The characteristics of this compartment are as follows:

1. The proximal-most, distinct compartment of this autophagic pathway is a collection of vacuoles (Golgi-associated vacuoles, GAV) near the trans-Golgi

2. The GAV are encased by cisternal membranes which appear to be continuous with ribosylated endoplasmic reticulum. These membranes resemble “isolation membranes” involved with initial sequestration of cargo to be autophagocytosed.

3. The GAV contains five type of electron-dense particles, proposed to represent different maturational intermediates of lipid donor and lipid acceptor particles. The same five types of particles are also seen within the secretory pathway (ie. the endoplasmic reticulum and the Golgi) but they show a different particle-particle and particle-membrane association.

4. Based on immunofluorescent studies, Map1LC3 (marker of all autophagic structures, but most strongly of early autophagocytic structures) and apolipoprotein B (protein component of very low density lipoproteins) co-localize in the GAV.

5. Dense vacuolar structures, with a more advanced degradative content which are reactive for the autofluorescent drug monodansylcadaverine, are located near the GAV.

Pharmaceutical Compositions and Methods of Treatment

In view of the inventors' discovery that autophagocytosis modulates TG and VLDL secretion, the invention encompasses the use of autophagocytosis modulating compounds for modulating serum levels of TG and/or VLDL and the use of autophagocytosis modulating compounds for the preparation of medicaments useful for treating diseases or disorders characterized by abnormal levels of TG and/or VLDL.

Pharmaceutical Compositions Useful for Reducing Serum Levels of TG and VLDL

In one aspect, the present invention provides the use of autophagocytosis inducing compounds for the production of pharmaceutical compositions useful for reducing serum levels of triglycerides and/or VLDL.

Pharmaceutical compositions of according to the present invention useful for reducing serum levels of triglycerides and/or VLDL comprise an autophagocytosis inducing compound and a pharmaceutically acceptable carrier.

The term “autophagocytosis inducing compound” encompasses small organic molecules, peptides, proteins, antibodies, antibody fragments, and nucleic acid sequences including DNA and RNA sequences which are capable of promoting autophagocytosis, and in particular, the maturation of autophagosomes to autophagolysosomes.

For example, the autophagocytosis inhibiting compound may be an antisense DNA or RNA molecule engineered to inhibit transcription or expression of proteins which inhibit or down regulate autophagocytosis. For example, the autophagocytosis inducing compound may be an antisense sequence designed to block transcription or expression of Class I P13′kinase, a known inhibitor of autophagocytosis.

The autophagocytosis inducing compound may be a recombinant DNA molecule which encodes for a protein which promotes induction/initiation of autophagocytosis. For example, the autophagocytosis inducing compound may be a recombinant DNA molecule encoding for an autophagocytosis agonist such as Map1LC3, GABARAP, GATE16, or Class III P13′ kinase.

The autophagocytosis inducing compound may be an antibody or antibody fragment which selectively recognizes and binds to proteins which inhibit or down regulate autophagocytosis. For example, the autophagocytosis inducing compound may be an antibody which binds to Class I P13′kinase.

The autophagocytosis inducing compound may be a recombinant DNA molecule which encodes for a protein which promotes induction/initiation of autophagocytosis. For example, the autophagocytosis inducing compound may be a recombinant DNA molecule encoding for an autophagocytosis agonist such as be Map1LC3 (microtubule associated protein 1 light chain 3/LC3), GABARAP (γ-aminobutyric acid (GABA)_{A}-receptor-associated protein), GATE16 (Golgi-associated ATPase enhancer of 16 kDa) and Class III P13′kinase. These proteins have been identified as agonists for the induction/initiation of the autophagocytosis in yeast (Mizushima et al., 2003, Int. J. Biochem. and Cell Biology 35, 553-561) and mammalian cells. Isoforms of each the preceding proteins may be used to prepare the pharmaceutical compositions according the invention. For example, Map1LC3 exists in two isoforms in the rat (I and II) and in three isoforms in humans, A, B and C.

Alternatively, the autophagocytosis inducing compound may be a protein which promotes autophagocytosis such as, but not limited to be Map1LC3, GABARAP, GATE16, and Class III P13′kinase.

It is thought that both Map1LC3 and its' yeast analogue become covalently attached to PE moieties within the membrane of autophagic membranes. Thus, compounds which alter the amount/concentration of PE in the membrane are useful as autophagocytosis inducing compounds for the preparation of pharmaceutical compositions according to the invention. The autophagocytosis inducing compounds may be prepared in pharmaceutical compositions comprising other anti-lipid or cardiovascular agents.

Pharmaceutical Compositions Useful for Increasing Serum Levels of TG and VLDL

In another aspect, the present invention provides the use of autophagocytosis inhibiting compounds for the preparation of a pharmaceutical composition useful for increasing serum levels of TG and/or VLDL. The pharmaceutical composition of the invention comprises an autophagocytosis inhibiting compound and a pharmaceutically acceptable carrier. The term “autophagocytosis inhibiting compound” encompasses small organic molecules, peptides, proteins, antibodies, antibody fragments, and nucleic acid sequences including DNA and RNA sequences which are capable of inhibiting autophagocytosis entirely or in part.

In a preferred embodiment of the invention, the autophagocytosis inhibiting compound is wortmannin, 3-methyladenine or LY294002 which are known inhibitors of autophagocytosis and inhibit phosphatidylinositol 3′kinases (PI3′kinases).

Rapamycin is a known inhibitor of autophagocytosis and may also be used to prepare the pharmaceutical composition according to the invention. Rapamycin is a macrocyclic lacton which inhibits function of mTor (mammalian rapamycin target) a Ser/Thr kinase with homology to PI3′kinases. Class I PI3′kinases are also known autophagocytosis antagonists and may be used as the autophagocytosis inhibiting compound to prepare the pharmaceutical composition of the invention.

Preparation and Administration of Pharmaceutical Compositions

The pharmaceutical compositions of the present invention may be manufactured in a manner that is itself known, e.g., by means of conventional mixing, dissolving, granulating, dragee-making, levigating, emulsifying, encapsulating, entrapping orlyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills; dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/orpolyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The pushfit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed.

Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients.

Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

One may administer the drug in a targeted drug delivery system, for example, in a liposome coated with an antibody specific for affected cells. The liposomes will be targeted to and taken up selectively by the cells.

The pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. In therapy or as a prophylactic, the active agent may be administered to an individual as an injectable composition, for example as a sterile aqueous dispersion, preferably isotonic. A therapeutically effective dose further refers to that amount of the compound sufficient to result in amelioration of symptoms associated with such disorders. Techniques for formulation and administration of the compounds of the instant application may be found in Mack E. W., 1990, Remington's Pharmaceutical Sciences, Mack Publishing Company, Easton, Pa., 13^{th} edition. For administration to mammals, and particularly humans, it is expected that the daily dosage level of the active agent will be from 0.001 mg/kg to 10 mg/kg, typically between 0.01 mg/kg and 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

Method of Treatment

The present invention encompasses the use of autophagocytosis modulating compounds for altering serum levels of triglycerides and VLDL.

In one aspect, the invention provides the use of autophagocytosis inducing compounds for reducing serum levels of triglycerides and VLDL. In another aspect, the invention provides the use of autophagocytosis inducing compounds for treating or preventing disorders resulting from or associated with elevated serum levels of triglycerides and/or VLDL.

The reduction of serum levels of triglycerides and VLDL and the treatment or prevention of disorders resulting from or associated with elevated serum levels of triglycerides and/or VLDL may be accomplished by administering a therapeutically effective amount of an autophagocytosis inducing compound to a patient in need thereof.

Diseases and disorders which may be treated or prevented by administering an autophagocytosis inducing compound include, but are not limited to: hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia, or combinations thereof. Generally, autophagocytosis inducing compounds and pharmaceutical compositions thereof are useful for treating patients having a disorder which would benefit in the reduction of serum levels of TG and/or VLDL.

By an “effective amount” or a “therapeutically effective amount” of a pharmacologically active agent is meant a nontoxic but sufficient amount of the drug or agent to provide the desired effect. In a combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The therapeutic effective amount of any of the active agents encompassed by the invention will depend on number of factors which will be apparent to those skilled in the art and in light of the disclosure herein. In particular these factors include: the identity of the compounds to be administered, the formulation, the route of administration employed, the patient's gender, age, and weight, and the severity of the condition being treated and the presence of concurrent illness affecting the gastrointestinal tract, the hepatobillary system and the renal system. Methods for determining dosage and toxicity are well known in the art with studies generally beginning in animals and then in humans if no significant animal toxicity is observed. The appropriateness of the dosage can be assessed by monitoring lipid levels. Where the dose does not improve serum TG and/or VLDL levels following at least 1 to 10 weeks of treatment, the dose can be increased.

Where the autophagocytosis inducing compound to be administered is in the form of a nucleic acid sequence such as a DNA or RNA sequence, conventional gene therapy approaches may be employed. The administration of autophagocytosis inducing compounds in the form of DNA or RNA sequences can be accomplished using methods known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Conventional pharmaceutical therapies may be employed for the administration of an autophagocytosis inducing compound in the form of a small organic molecule, a pharmacological compound or agent, a peptide, a protein, an antibody or an antibody fragment. The active ingredient can be administered with a suitable pharmaceutical carrier as discussed above.

In a preferred embodiment of the invention, the treatment of prevention of disorders resulting from or associated with elevated serum levels of triglycerides and/or VLDL is accomplished by administering a therapeutically effective amount of Map1LC3, GABARAP, GATE16, Class III P13′ kinase or a combination thereof.

Thus, disorders treatable by the compositions of the present invention include hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia or combination thereof.

Methods of Identifying Autophagocytosis Modulating Compounds and Uses of Identified Compounds

The invention includes methods for screening nucleotides, proteins, compounds or pharmacological agents, which either enhance or inhibit autophagocytosis. Cell based, cell lysate and/or purified enzyme assays can be used to identify these enhancing or inhibiting compounds. As used herein, the term “test compound” includes but is not limited to small molecules (e.g. small organic molecules), pharmacological compounds or agents, peptides, proteins, antibodies or antibody fragments, and nucleic acid sequences, including DNA and RNA sequences.

In one aspect, the present invention provides a method identifying autophagocytosis modulating compounds which involves assaying for changes in lipid degradation and secretion. The method comprises the steps of: (a) providing a control cell culture system and a test cell culture system; (b) administering a test compound to cells in said test cell culture system; and (c) assaying for autophagocytosis markers in said control cell culture system and said test cell culture system, wherein an abnormal value for said autophagocytosis markers in said test cell culture system as compared to said control cell culture system indicates that the test compound modulates autophagocytosis.

In an embodiment of the invention, the autophagocytosis markers are VLDL or VLDL precursors. In a further embodiment of the invention, the VLDL precursors assayed include PC moiety containing lipids and PE moiety containing lipids. In a further preferred embodiment the PC moiety containing lipid is 18:1(n-9) PC and the PE moiety containing lipid is 20:5(n-3) PE.

A compound is positively identified as being an autophagocytosis modulator if the levels of VLDL and VLDL precursors in the ER and Golgi cell fractions and in the culture medium for the test cell culture, are abnormal as compared to untreated control cell culture. A test compound is identified as being an autophagocytosis inducing agent if: (1) the levels of VLDL and VLDL precursors found in the ER and Golgi fractions are higher than the levels observed for the untreated control cells and (2) the levels of VLDL and VLDL precursors in the cell medium are lower than the levels observed for the untreated control cells. Conversely, a test compound is identified as being an autophagocytosis inhibiting agent if: (1) the levels of VLDL and VLDL precursors found in the ER and Golgi fractions are lower than the levels observed for the untreated control cells and (2) the levels of VLDL and VLDL precursors in the cell medium are higher than the levels observed for the untreated control cells.

The VLDL and VLDL precursors can be assayed using known chromatographic methods known in the art such high performance liquid chromatography and more preferably known mass spectrometry methods.

In another aspect, the invention provides a method for identifying autophagocytosis inducing compounds involving the examination of changes of membrane composition. The method comprises the steps of: (a) administering a test compound to cells in a cell culture system; and (b) assaying for PC moiety containing lipids and PE moiety containing lipids in ER and Golgi cell fractions. A test compound is identified as an autophagocytosis inducing compound if there is a decrease in levels of PC moiety containing lipids and an increase PE moiety containing lipids as compared to untreated control test cells. In an embodiment of the invention, the PC moiety containing lipid assayed is 18:1(n-9) PC and the PE moiety containing lipid assayed is 20:5(n-3) PE. The PE and PC moiety containing lipids can be assayed using known mass spectrometry techniques.

In another embodiment, the autophagocytosis biomarkers are apoB100 and Map1LC. The biomarkers can be assayed using immunofluorescence to determine the degree of co-localization of apoB100 and Map1LC. A test compound is identified as an autophagocytosis modulator if the degree of co-localization of apoB100 and Map1LC3 is abnormal as compared to untreated control cells. A test compound is identified as being an autophagocytosis inducing agent if the degree of co-localization is greater than that observed for untreated cells. Conversely, a test compound is identified as being an autophagocytosis inhibiting agent if there is no co-localization or the degree of co-localization is less than that observed for untreated cells.

Cell culture systems useful for practicing any of the methods of the invention include fungal or mammalian cell lines In an embodiment of the invention, the cells may be hepatocytes and hepatoma cells. More preferably, the cells are rat hepatocytes or hepatoma cells which stably express the human apoB100 protein. The expressed apoB100 protein may be a tagged fusion protein which facilitates detection and measurement of the protein. For example, methods according to the invention may be practiced using McA-RH-7777 cells which express fluorescent tagged apoB100. Such stable cell lines can be used to screen chemical derivatives of initial hits, titrate optimal dosages and screen libraries of commercially available molecules The apoB100 fusion protein can also be prepared using other tags known in the art in addition to fluoroscent tags. For example, the apoB1000 protein can be tagged with tetra-cysteine-Cys-Cys-X-X-Cys-Cys-(wherein X is any amino acid). Tetra-cysteine tagged proteins can be assayed using the bi-arsenical-tetra-cysteine detection method (Zhang et al., 2002, Nar. Rev. Mol. Cell. Biol. 3, 906-918)

Autophagocytosis inducers identified using the methods of the invention can be used to prepare pharmaceutical compositions useful for reducing serum levels of TG and VLDL. Such identified compounds would also be useful for treating and preventing diseases and disorders which would be benefit from a reduction of serum levels of TG and VLDL such as, but not limited to: hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia or combinations thereof.

Conversely, autophagocytosis inhibitors identified using the methods of the invention can be used to prepare pharmaceutical compositions useful for treating and preventing diseases and disorders which would benefit from an increase in serum levels of TG and VLDL such as but not limited to: irritable bowel syndrome and Crohn's disease.

It is understood that the present invention is not limited to the particular methodology, protocols, cell lines, and reagents described herein. Generally, the laboratory procedures in cell culture and molecular genetics described below are those well known and commonly employed in the art.

Standard techniques are used for recombinant nucleic acid methods, polynucleotide synthesis, microbial culture, transformation, transfection, etc. Generally, enzymatic reactions and purification steps are performed according to the manufacturer's specifications. Although any methods and materials similar or equivalent to those described herein can be used in the practice or testing of the present invention, the selected methods, devices, and materials are described below.

### EXAMPLE EXPERIMENTAL PROCEDURES

Materials—Glycerol [¹⁴C]trioleate (57 mCi/mmol), [³H]glycerol (1.1 Ci/mmol), [¹⁴C]oleic acid (55 mCi/mmol), [³⁵S]methionine/cysteine (1000 Ci/mmol), Protein A Sepharose™ CL-4B beads, and HRP-linked anti-mouse or anti-rabbit IgG antibodies were purchased from Amersham Pharmacia Biotech. [³H]Eicosapentaenoic acid (150 Ci/mmol) was purchased from American Radiolabeled Chemicals, Inc. Fibronectin, monodansylcadaverine and oleic acid were obtained from Sigma. Triglyceride, and phospholipid standards were from Avanti Polar Lipids. Eicosapentaenoic acid (peroxide free) was from Cayman. Monoclonal anti-human apoB antibody 1D1 was a gift of R. Milne and Y. Marcel (University of Ottawa Heart Institute). Polyclonal anti-MTP and anti-rat apoA1 antisera were gifts of C. C. Shoulders (Hammersmith Hospital, United Kingdom) and J. E Vance (University of Alberta, Canada), respectively. The anti-rat Map1LC3 antiserum was kindly provided by A. Nara and T. Yoshimori (National Institute of Genetics, Mishima, Japan). Polyclonal antiserum against human LDL was produced in our laboratory. Protease inhibitor cocktail and chemiluminescent blotting substrate was purchased from Roche Diagnostics. Culture plate inserts (0.4 μm MILLICELL™-CM, 30-mm diameter) were purchased from Millipore.

Cell Culture and Fatty Acid Treatments—Transfected McA-RH7777 cells stably expressing human apoB100 (McLeod et al., 1994, J. Biol. Chem. 269, 2852-2862) were cultured in Dulbecco's modified Eagle's medium (DMEM) containing 10% fetal bovine serum (FBS), 10% horse serum and 200 μg/ml G418. Routinely, the cells were incubated with 0.4 mM fatty acids for 16-18 h in the presence of 20% FBS prior to experiments. During experiments, the cells were kept in fresh medium containing 20% FBS plus other reagents as indicated in the figure legends.

Pulse-chase Experiments—In pulse-chase experiments where secretion efficiency of apoB was determined, cells were cultured in 60-mm dishes to 80% confluency, and preincubated with 0.4 mM oleate or EPA for 16 h. The cells were labelled with [³⁵S]methionine/cysteine (100 μCi/ml in 1 ml methionine- and cysteine-free DMEM containing 20% FBS and 0.4 mM oleate or EPA) for 1 h and incubated with chase medium (DMEM containing 20% FBS and 0.4 mM oleate or EPA) for indicated times. ³⁵S-apoB100 secreted in the medium and associated with the cells was immunoprecipitated using polyclonal antiserum raised against human LDL and resolved by SDS-PAGE/fluorography as described (Tran et al., 2000). In pulse-chase experiments where apoB100 in the membrane and lumenal content of different subcellular fractions was determined, cells in 100-mm dishes were labelled with [³⁵S]methionine/cysteine (200 μCi/ml in 4 ml methionine- and cysteine-free DMEM containing 20% FBS and 0.4 mM oleate or EPA) for 20 min. The cells were then incubated with chase medium for 15, 30 and 45 min. At the end of each chase time, the medium was collected and subjected to cumulative rate flotation centrifugation (Wang et al., 1999) to resolve apoB100-VLDL₁ (S_{f}>100) and apoB100-VLDL₂ (S_{f} 20-100) from other lipoproteins (i.e. IDL, LDL and HDL). The ³⁵S-apoB100 in each fraction was recovered by immunoprecipitation. Also, at the end of each chase time, the radiolabeled cells were harvested in 2 ml of ice-cold homogenization buffer (10 mM Tris-HCl, pH 7.4, 250 mM sucrose, 5 mM EDTA, and serine/cysteine protease inhibitor mixture), mixed with two 100-mm dishes of unlabeled cells, homogenized by passing ten times through a ball-bearing homogenizer, and subjected to subcellular fractionation and carbonate-treatment as described below.

Subcellular Fractionation—Three subcellular fractions (i.e., ER, fractions 1 through 3; cis/medial Golgi, fractions 4 through 8; distal Golgi, fractions 9 through 15) were obtained from the cell lysates using Nycodenz gradient centrifugation (Hammond and Helenius, 1994, J. Cell Biol. 126, 41-52; Rickwood et al., 1982, Anal. Biochem. 123, 23-31) of the post-nuclear supernatant as previously described (Tran et al., 2002).

Analysis of ApoB100 Associated with Membranes and Lumenal Contents of Microsomes—Lumenal contents were separated from membranes by sodium carbonate treatment followed by centrifugation (Tran et al., 2002). The ³⁵S-labelled apoB100 proteins associated with the membrane and lumen were recovered by immunoprecipitation and analyzed by SDS-PAGE/fluorography as previously described (Tran et al., 2002).

Competitive Enzyme Linked Immunosorbent Assay (ELISA)—The ELISA plates were coated with human LDL (1 mg/ml in PBS, 16 h, 4° C.), blocked with skim milk (5% in PBS, 2 h, 37° C.), and washed three times with PBS containing 0.02% Tween-20. The plates were incubated with apoB monoclonal antibody 1D1 (1:64,000, 16 h, 4° C.) in the presence of serial diluted concentrations of human LDL or medium samples. The plates were washed and incubated with horseradish peroxidase-linked anti-mouse IgG antibody (1:10,000, 2 h, 37° C.), followed by addition of the liquid substrate system for ELISA (3,3′,5,5′-tetramethyl-benzidine). The reaction was quantified colorimetrically by spectrophotometer reading at OD₆₆₅.

Transmission Electron Microscopy—Cells were cultured in normal culture medium on MILLICELL™-CM insert membranes precoated with fibronectin for 20 h, and incubated for additional 4 h with fresh DMEM containing 20% FBS and 0.4 mM oleate or EPA. The samples were processed for transmission electron microscopy as previously described (Tran et al., 2002). Single and serial thin sections (silver-gold interference colors) were visualized in a Hitachi H-7000 transmission electron microscope, and captured at a range of negative magnifications (8,000-120,000 times). Panoramic tiling was used to capture large fields. The 3D model was prepared from Golgi fields from 7 consecutive serial images (positive magnification=70,000 times), by the method previously described (Thorne-Tjomsland et al., 1998, Anat. Rec. 250, 381-396), with the following modifications. The serial fields were scanned into Adobe Photoshop 5.5 of an Imac 700 MHz G4 computer. Alignment of consecutive sections by fiducial markers was carried out prior to object-contouring and -separation. Concatenation and volume rendering were done in Synu on an SGI-OS 2, and image capture was with Photoshop on a Macintosh platform. The diameter of electron-dense particles, which represent a combination of lipoprotein particles and lipid droplets, were measured in 40 randomly selected Golgi regions from EPA-treated cells. Negative magnification was 40,000 times and positives were further magnified three times. Measurements were from positives, using a digital caliper [technical specifications in required range (0-150 mm on positive): max resolution=0.01 mm; accuracy=0.02 mm; repeatability=0.01 mm]. The precision in our system was tested by measuring the diameters of each of two electron-dense particles (20 nm and 40 nm diameter) 40 times; SD for the average converted measurements was <1 nm. Criteria for selecting Golgi, establishing cis-trans polarity, and measuring lipid/lipoprotein particles were as described (Tran et al., 2002). Lipid/lipoprotein particles were classified as membrane-associated if directly apposed to the lumenal Golgi leaflet or with a membrane diverging from this, otherwise as lumenal.

Immunocytochemistry—Cells were plated onto fibronectin-precoated coverslips for 24 h, incubated with 0.4 mM oleate or EPA in DMEM containing 20% FBS for 4 h and fixed with 3% paraformaldehyde in PBS. Cells were permeabilized with 1% Triton X-100 in blocking buffer (10% FBS in PBS) for 30 min and probed with primary antibodies, i.e., monoclonal antibody 1D1 (1:1000) for human apoB and polyclonal antibody against rat Map1LC3 (1:200) for 1 h. Cells were then incubated with a mixture of secondary antibodies (1:200), i.e., of goat anti-mouse IgG conjugated with Alexa Fluor™488 (green) and goat anti-rabbit IgG conjugated with Alexa Fluor™594 (red) for 1 h. The coverslips were mounted onto glass slides using SlowFade AntiFade kits (Molecular Probes) and the images were captured by an MRC-1024 laser scanning confocal imaging system.

Monodansylcadaverine (MDC) Labelling—Cells were plated onto poly-d-lysine coated glass bottom microwell dishes (MatTek Co) for 24 h and incubated with 0.4 mM oleate or EPA in DMEM containing 20% serum for 4 h. Cells were then incubated with 0.05 mM MDC in DMEM at 37° C. for 10 min (Biederbick, 1995, Eur. J. Cell Biol. 66, 3-14; Munafo and Colombo, 2001, J. Cell Sci. 114, 3619-3629). After incubation, cells were washed three times with PBS and fixed in 3% paraformaldehyde for 30 min. After fixation, cells were washed four times with PBS and analyzed by fluorescence microscopy using an Olympus IX70 inverted microscope equipped with a 12 bit IMAGO SVGA CCD camera and the Till Polychrome IV monochrometer. MDC was exited at 380 nm using a fura filter set (T.I.L.L. Photonics GmbH). The images were processed using the TillVisION software, version 4.0.

Tandem Mass Spectrometry—Cells were kept in DMEM (20% FBS±0.4 mM oleate or EPA) for 16 h and re-incubated with fresh medium (20% FBS±0.4 mM oleate or EPA) for an additional 2 h. The membrane and lumen preparations from ER (Nycodenz fractions 1 through 3), cis/medial Golgi (fractions 4 through 8), and distal Golgi (fractions 9 through 15) were derived from cells pooled from eight 100-mm dishes. Lipids were extracted from the samples with chloroform/methanol/acetic acid/saturated NaCl/H₂O (4:2:0.1:1:2, by volume) in the presence of 230 pmol dimirystoyl (14:0-14:0) PC and 110 pmol dipalmitoyl (16:0-16:0) PE as internal standards. Aliquots of lipid extracts were applied to tandem mass spectrometry, and the molecular species (i.e. fatty acid composition) of PC and PE was determined by daughter ion analysis in the negative ion mode as previously described (Tran et al., 2002; DeLong et al., 1999, J. Biol. Chem. 274, 29683-29688). The integrated area under the peak of each molecular species was quantified by comparing with those of internal standards.

Other Assays—The TG transfer activity of MTP was determined according to published method (Wetterau et al., 1992, Science 258, 999-1001) with modifications (Wang et al., 1999). The phosphatidate phosphohydrolase activity was determined by an established method (Jamal et al., 1991, J. Biol. Chem. 266, 2988-2996). Lipid extraction and analysis by TLC was performed as previously described (Tran et al., 2000). Protein was determined using the BCA™ protein assay kit (Pierce).

### Example 1—EPA Treatment Decreases TG Secretion

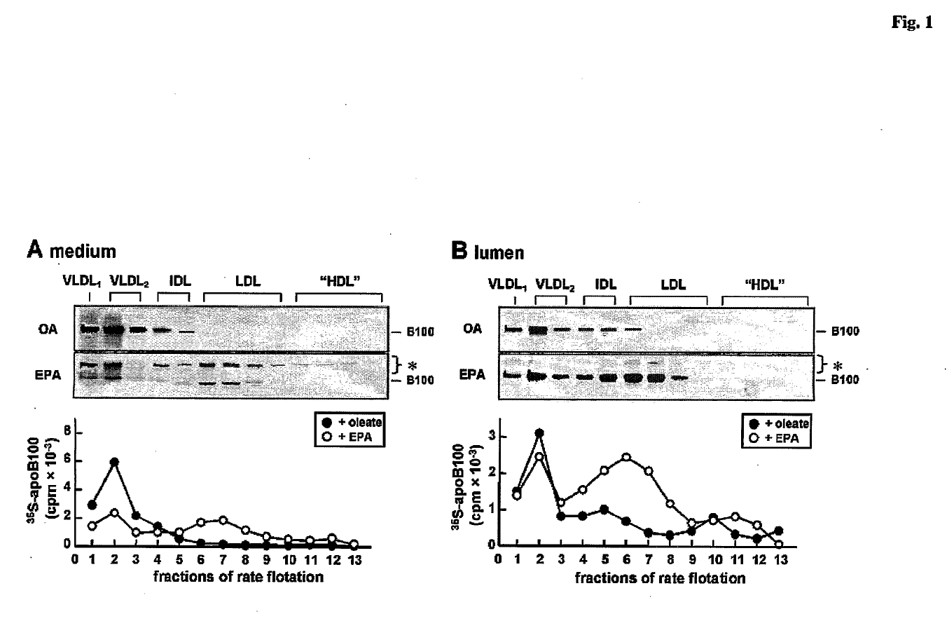

Cells pretreated with oleate or EPA for 16 h were labelled with [³⁵S] methionine/cysteine for 30 min and cultured with normal media (chase) for 1 h. The conditioned media (FIG. 1A) or lumenal contents of microsomes (FIG. 1B) were subjected to rate flotation centrifugation. The [³⁵S]-apoB100 in each fraction was immunoprecipitated, resolved by polyacrylamide gel electrophoresis in the presence of SDS (SDS-PAGE), and visualized by fluorography. The top panels of FIGS. 1A and 1B are representative fluorograms of experiments that were performed more than three times with similar results. The bands marked with asterisks represent [³⁵S] apoB100 species that are insoluble in the presence of SDS. The bottom panels of FIGS. 1A and 1B set out the radioactivity associated with [³⁵S] apoB100 in each fraction (including the insoluble species) was quantified. “HDL” as indicated in the top panels of FIGS. 1A and 1B, refers to LpB whose buoyant density resembles that of plasma HDL.

Previous studies with man (Fisher et al., 1998, J. Lipid Res. 39: 388-401; Hsu et al., 2000, Am. J. Clin. Nutr. 71: 28-35; Sullivan et al., 1986, Atherosclerosis, 61: 129-134;) and monkeys (Parks et al., 1989, J. Lipid Res. 30: 1535-1544; Parks et al., 1990, J. Lipid Res. 31: 455-466) have shown that EPA treatment reduces the plasma VLDL-apoB100 and VLDL-TG concentration. In normolipidemic and hyperlipidemic human subjects, fish oil diet decreased plasma TG and VLDL-apoB but increased LDL-apoB and LDL-cholesterol whereas total plasma apoB concentration did not change (Nestel et al., 1984, J. Clin. Invest. 74: 82-89; Fisher et al., 1998, J. Lipid Res. 39: 388-401). In men with visceral obesity, n-3 fatty acid supplementation decreased VLDL-apoB production rate by 29% (Chan et al., 2003, Am. J. Clin. Nutr. 77: 300-307). These data suggest that the specific target of fish oil is probably the assembly of large, TG-rich apoB-containing lipoproteins (LpB). It was hypothesized that EPA treatment might exert an inhibitory effect on the second-step assembly of VLDL, and tested this hypothesis using human apoB100 transfected McA-RH7777 cells as a model. The temporal and spatial events associated with VLDL assembly and secretion between oleate and EPA treatment conditions were contrasted. In all experiments described below, the cells were cultured in media supplemented with 20% serum to minimize proteasome-mediated intracellular degradation of newly synthesized apoB100 and facilitate exogenous oleate-induced VLDL assembly (McLeod et al., 1996, J. Biol. Chem. 271: 18445-18455). Cells were pulse-labelled with [³⁵S] amino acids for 30 min, and apoB100 associated with lipoproteins either secreted into the medium or present within the lumen of microsomes (after carbonate treatment) were determined at the end of 1-h chase. The amount of [³⁵S] incorporated into apoB100 at the end of a 30 min pulse was identical between oleate- and EPA-treated cells (data not shown). At the end of 1-h chase, EPA treatment decreased (by 50%. as compared to oleate-treatment) [³⁵S]-apoB100 in VLDL (VLDL₁ and VLDL₂) in the media (FIG. 1A) and increased [³⁵S]-apoB100 (by six-fold) in fractions of high density [e.g. intermediate density lipoproteins (IDL) and LDL] in the microsomal lumen (FIG. 1B). The difference in lumenal VLDL-associated apoB100 between oleate- and EPA-treated cells was less remarkable than that of secreted VLDL (FIG. 1B), indicating that the ability to assemble some VLDL was retained in EPA-treated cells.

Unexpectedly, there were markedly increased [³⁵S]-apoB100 species, found in microsomal lumen (and in the medium as well) of EPA-treated cells, that were insoluble in SDS sample buffer (bands marked by asterisks in FIGS. 1A and B). The retarded band reacted with antibody 1D1 recognizing human apoB (data not shown). Inclusion of 6% urea during SDS-PAGE was unable to eliminate apoB100 aggregation (data not shown). Treatment of the sample with either water-soluble (e.g. butylated hydroxytoluene) or lipid-soluble anti-oxidants (e.g. α-tocopherol) also failed to prevent apoB100 aggregation (data not shown). The nature of these apparently aggregated apoB100 species detected in the microsomal lumen and medium of EPA-treated cells is unclear; they may represent assembly intermediates accumulated within the secretory pathway (see below). When the total amount of secreted [³⁵S]-apoB100 was quantified (i.e. the sum of [³⁵S]-apoB100 in all fractions including the aggregated species), it showed similar secretion between EPA- and oleate-treated cells. Moreover, quantification of apoB100 proteins by competitive ELISA showed that the amount of apoB100 protein accumulated in the medium after 16-h incubation decreased slightly from EPA-treated cells as compared to oleate-treated cells (oleate, 2.74±0.48; EPA, 2.5±0.43 μg/ml), but the difference did not reach statistical significance (p>0.05, n=3). Metabolic labelling of lipids with [³H] glycerol showed 50% reduction in secretion of [³H] glycerol-labelled TG from EPA-treated cells, although incorporation of [³H] glycerol into cellular TG was higher in EPA—than in oleate-treated cells. The incorporation of [³H] glycerol into secreted PC was not affected (Table I).

TABLE I
Synthesis and secretion of [³H]glycerol-labelled TG and PC

| | [³H]TG | | [³H]PC | |
|---|---|---|---|---|
| | Medium | Cell | Medium | Cell |
| | cpm × 10⁻³/dish^{a} | | | |
| Oleate | 12.87 ± 1.59 | 33.80 ± 0.62 | 4.10 ± 0.12 | 15.00 ± 0.23 |
| EPA | 7.18 ± 0.96^{b} | 40.84 ± 0.70^{b} | 3.86 ± 0.57 | 15.80 ± 0.44 |

^{a}Radioactivity associated with [³H]PC and [³H]TG at the end of 2-h labelling with [³H]glycerol in the presence of oleate or EPA was determined. Data are means ± SD of triplicate determination.
^{b}p < 0.05, compared to oleate-treated cells.

Together, data from these cell culture experiments, in agreement with in vivo studies (Nestel et al., 1984, J. Clin. Invest. 74: 82-89), indicate that EPA treatment results in reduced secretion of TG with marginal decrease in the amount of apoB100 secreted.

### Example 2—EPA Treatment Promotes Post-ER Degradation of ApoB100

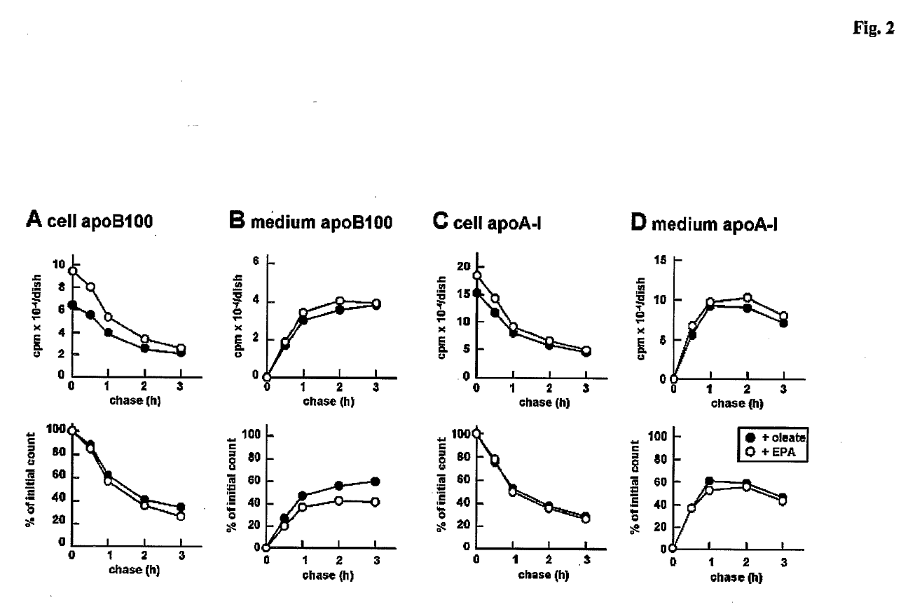

Cells pretreated with oleate and EPA were labelled with [³⁵S]methionine/cysteine for 1 h and chased for up to 3 h. Oleate and EPA were present in both pulse and chase media. The [³⁵S]-apoB100 from total cell lysates (FIG. 2A) or conditioned media (FIG. 2B) was immunoprecipitated, resolved by SDS-PAGE, and visualized by fluorography. Radioactivity associated with [³⁵S]-apoB100 was quantified. As shown in the top panels of FIG. 2A to 2B, the data is expressed as absolute amount of radioactivity associated with [³⁵S]-apoB100 at the end of 1-h pulse. As shown in the top panels of FIG. 2A to 2B, the data is expressed as percent of the initial counts associated with ³⁵S-apoB100 at the end of 1-h pulse. As shown in FIGS. 2C and 2D, the radioactivity associated with [³⁵S]-apoA-I in the cells (FIG. 2C) and medium (FIG. 2D) was similarly quantified. The experiments were repeated and similar results were obtained.

It has been shown previously that VLDL, particles carry >80% of total TG but <10% of total apoB100 secreted from oleate-treated McA-RH777 cells (Wang et al., 1999, J. Biol. Chem. 274: 27793-27800). Thus the possibility was considered that the above pulse (30-min)-chase(60 min) experiment might fail to detect decreased secretion and increased post-translational degradation of apoB100 because n-3 fatty acid treatment was reported to selectively decrease apoB100 in VLDL fractions (Fisher and Ginsberg, 2002, J. Biol. Chem. 277: 17377-17380). In the next set of experiments, the pulse-labelling period was extended to 1 h to maximize [³⁵S]-labelling of apoB100 and to allow examination of potential posttranslational degradation. Under these conditions, the amount of [³⁵S] incorporated into apoB100 at the end of 1-h pulse in EPA-treated cells (9.43×10⁴ cpm/dish) was ˜40% greater than in oleate-treated cells (6.40×10⁴ cpm/dish) (FIG. 2A, top). The high labelling of apoB100 at the end of 1-h pulse may reflect increased intracellular accumulation of newly synthesized apoB100 and/or impaired secretion. At the end of 3-h chase, the amount of cell-associated [³⁵S]-apoB100 in EPA-treated cells had decreased to levels comparable to those of oleate-treated cells (FIG. 2A, top), but notably the excess cell-associated [³⁵S]-apoB100 seen after 1-h pulse was not recovered in the medium during chase (FIG. 2B, top). The secretion efficiency, which measures the proportion of total metabolically labelled [³⁵S]-apoB100 secreted at the end of chase was decreased from 60% to 40% in EPA-treated cells compared to OA-treated cells (FIG. 2B, bottom). The cell-associated [³⁵S]-apoB100 at the end of chase was also slightly lower as compared with oleate treatment (FIG. 2A, bottom). In the same experiments, synthesis or secretion of apoA-I were relatively unaffected by EPA-treatment (FIGS. 2C & 2D). These kinetic studies suggest that in EPA-treated McA-RH7777 cells, a proportion of newly synthesized apoB100 was first retained intracellularly, then degraded through a mechanism which was less rapid than proteasome-mediated ER degradation. Rather, degradation of apoB100 in EPA-treated cells likely was achieved through a slow process similar to the previously reported post-ER mechanism (Fisher and Ginsberg, 2002, J. Biol. Chem. 277: 17377-17380).

### Example 3—EPA Treatment does not Effect ApoB100 Trafficking Through the ER or Proximal Golgi

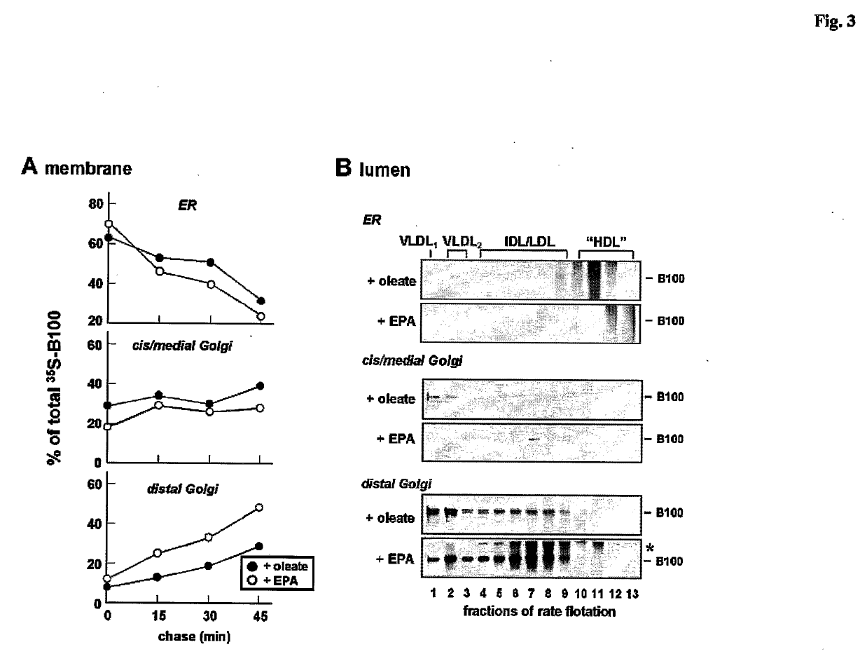

Cells pretreated with oleate or EPA were pulse labelled with [³⁵S]methionine/cysteine for 20 min and chased from 0-45 min. The subcellular compartments were fractionated by Nycodenz gradient centrifugation, and membranes (FIG. 3A, at various chase times) and lumenal content (FIG. 3B, at 45 min chase) of ER, cis/medial Golgi, and distal Golgi were isolated by sodium carbonate treatment followed by ultracentrifugation. The ³⁵S-apoB100 was immunoprecipitated and resolved by SDS-PAGE/fluorography as described in the “Experimental Procedures”. As shown in FIG. 3B, bottom panel, the bands marked with an asterisk represents ³⁵S-ApoB100 species which are insoluble in the presence of SDS.

Recent studies have shown that ER exit of apoB100 represents an important step in VLDL assembly (Gusarova et al., 2003, J. Biol. Chem. 278: 48051-48058). To determine if the accumulation of apoB100 which occurs in EPA-treated cells during pulse is due to altered apoB100 exit or its ER-to-Golgi trafficking, pulse-chase analysis was combined with subcellular fractionation experiments. The inventors showed previously that in McA-RH7777 cells, the newly synthesized apoB100 were mainly associated with the membranes of the ER/Golgi compartments (Tran et al., 2002, J. Biol. Chem. 277: 31187-31200). The rate at which the membrane-associated [³⁵S]-apoB100 exited the ER (calculated from four chase time points (i.e. 0, 15, 30 and 45 min) was higher in EPA-treated cells (−1.29±0.38% of total/min) than in oleate-treated cells (−0.59±0.17% of total/min)(p<0.05) (FIG. 3A, top panel). Likewise, the rate at which the membrane-associated [³⁵S]-apoB100 appeared in the distal Golgi was significantly higher in EPA-treated cells (0.83±0.10% of total/min) than in oleate-treated cells (0.39±0.11% of total/min) (p<0.05) (FIG. 3A, bottom panel). The rates with which [³⁵S]-apoB100 transited through the cis/medial Golgi were similar between the two treatments (FIG. 3A, middle panel). Thus, our data provides evidence that neither impaired ER exit nor a slowing in trafficking through the proximal Golgi, could explain the cellular accumulation of apoB100 at the end of a 1-h pulse in EPA-treated cells.

At the end of 45-min chase, augmented [³⁵S]-apoB100 was detected in the distal Golgi membrane (FIG. 3A), coupled with a pronounced accumulation of [³⁵S]-apoB100 in the lumenal fraction of distal Golgi (after carbonate treatment) in EPA-treated cells, the majority of which was associated with IDL/LDL fractions (FIG. 3B). This accumulation of apoB100 in the distal-Golgi membrane and lumen at least partially explains the cellular accumulation of apoB100 following a 1-h pulse in EPA-treated cells (FIG. 1A, top panel). These findings suggest the increased presence of assembly intermediates in EPA-treated cells. EPA-treatment did not affect the activities of either phosphatidate phosphohydrolase-1 or MTP (data not shown), ruling out that impaired VLDL assembly is attributable to attenuated TG synthesis of MTP-mediated TG-transfer in EPA-treated cells. Thus, the results show that EPA likely exerts its inhibitory effect on VLDL secretion within and/or downstream of the distal Golgi.

### Example 4—Size Distribution of Particles within the cis- and trans-Golgi of EPA-Treated Cells

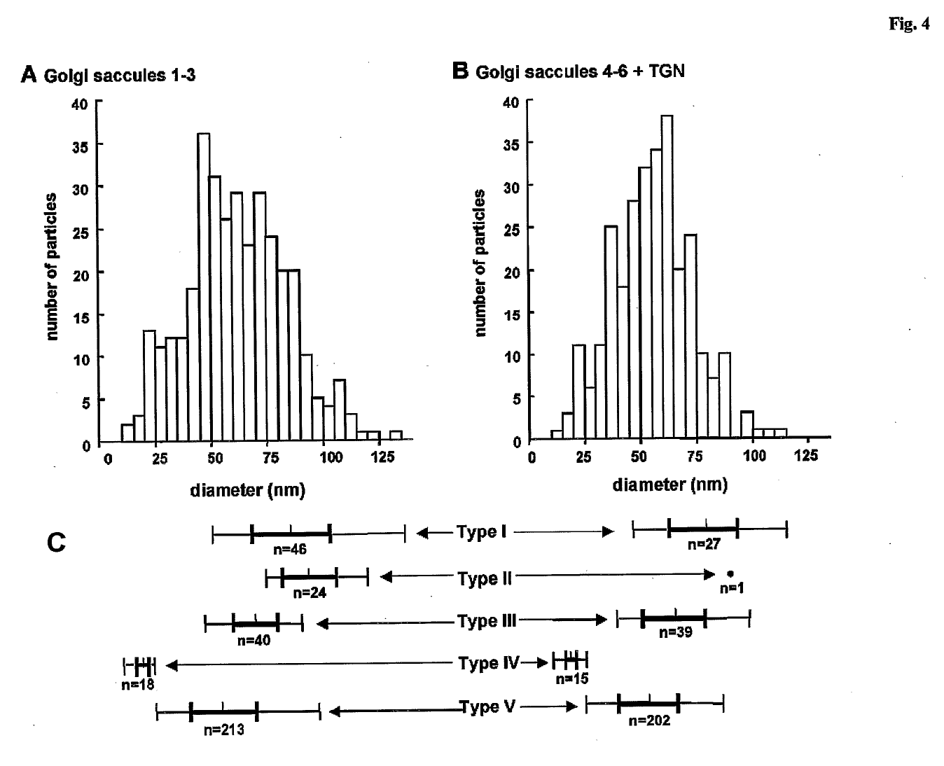

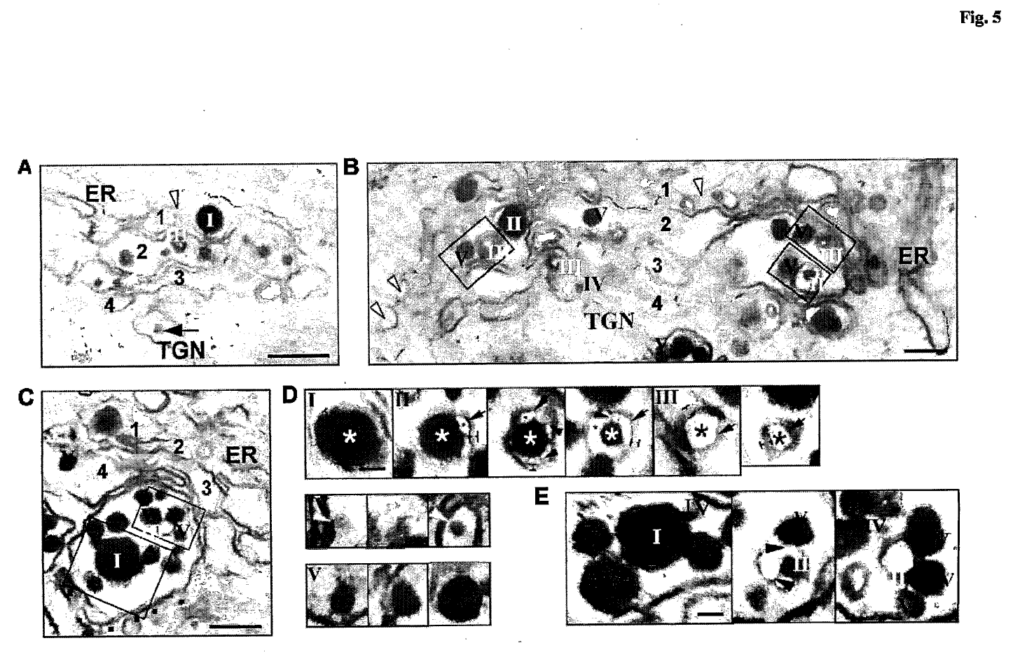

As shown in FIGS. 4A and 4B, the histograms depict the diameters of pooled particles within Golgi saccules 1-3 (cis-Golgi) (A) and saccules 4-6 (trans-Golgi)+TGN (B). As shown in FIG. 4C, for each of five identified particle types, i.e. Types I-V (for classification scheme, see FIG. 5D), the range of particle diameter (thin brackets) and the values for the average diameter (tick mark) ±1 SD (thick brackets) is plotted, using the values on the x-axis of the histogram.

Lipoprotein and lipid particles within the distal secretory compartments were analyzed by TEM to determine whether impaired second-step VLDL assembly was associated with generation of morphologically altered VLDL assembly intermediates. In McArdle cells treated with exogenous oleate to stimulate VLDL assembly and secretion. Lipoproteins with average diameters of 40±17 nm were observed in Golgi saccules 1-6, and a small number of electron-dense particles with diameter >80 nm were observed within Golgi saccules 4-6 (i.e. the trans-side of the Golgi) plus trans-Golgi network (TGN) (Tran et al., 2002, J. Biol. Chem. 277:25023-25030]. In EPA-treated cells, the population of these >80 nm particles was greatly increased in both the cis-(saccules 1-3) and trans-end (saccules 4-6) of Golgi plus TGN (FIG. 4A-B, histograms). [See legend of FIG. 4A below for detailed identification of cis-trans polarity of Golgi saccules]. A significant number of these >80 nm particles were comprised of particles characterized as either Type I, II or III (FIG. 4C) based on morphological features evident when the particles were viewed in situ in the Golgi (FIGS. 5A-C), including at higher magnification.

### Example 5—Five Types of Lipid/Lipoprotein Particles Identified in the Golgi and Associated Vacuoles

The Golgi stacks shown in panels A, B, and C, have 4 saccules (labelled 1 through 4). Saccule 1 has characteristic perforations (arrowheads). A trans-Golgi network (TGN) is shown in panels A and B, and in panel C, a large trans-Golgi associated vacuole (GAV) is shown which is partially encased by cisternal membranes (dotted line). Five types of particles, designated I though V, are present in the Golgi, including the TGN, and in the GAV. Higher magnification images of the five types of particles are shown in panel D. The putative proteinaceous coat (brackets) and core (white asterisks) of Type I-III particles is indicated as is the phospholipid monolayer between them (arrows). In Type II particles, thin strands of material span porosities (small asterisks) between the core and the phospholipid monolayer (arrowheads). Type IV particles (white arrowheads) and Type V particles represent respectively HDL- and VLDL-sized structures. Note in panel A, two Type IV particles (white arrows) in saccule 1 and 3 are membrane-associated whereas one Type IV particle (black arrow) in the TGN is lumenal. In Golgi saccules, Type I-V particles occur either singly, or in pairs (boxes in panel B), whereas in GAV the particles are frequently seen in clusters (boxes in panel C), which in higher magnification views (E) are comprised of a single Type I, II or III particle surrounded by several Type IV and V particles (left, middle, right panel respectively)

Based on the morphometric findings, a significant proportion of the >75 nm particles were categorized as either Type I, II or III (FIG. 4, bottom), based on distinctive morphological features evident when the particles were viewed in situ in the Golgi (FIGS. 5A-C), including at higher magnification (FIG. 5D). Particles with Type I-III morphologies were also seen in the tubular smooth ER and in the cytoplasm, within groups of cytoplasmic lipid droplets (not shown). The average size of Type I particles (100 nm) in the cis-most Golgi saccule of EPA-treated cells was at the low end of the range measured for cytoplasmic lipid droplets (0.1-50 μm) (Murphy and Vance, 1999, Trends Biochem. Sci. 24: 109-115) and their morphology (FIG. 5D, top panel) was similar to that of cytoplasmic lipid droplets, which regardless of size, have an electron-dense TG core surrounded by a phospholipid monolayer and a proteinaceous halo (Blanchette-Mackie et al., 1995 J. Lipid Res. 36:1211-1226). Hence Type I particles likely correspond to apoB-free lipid particles, such as those detected in the SER which are non-reactive for apoB by HRP-immunocytochemistry (Alexander et al., 1976. J. Cell Biol. 69:241-263). Type II and III particles display a partial and absent core respectively (FIG. 5D, top panel) and may correspond to partially and fully delipidated lipid particles. Accumulation of lipid-particles in the secretory pathway is compatible with the lipid partitioning experiments described below. These data present morphological evidence that lipid droplets accumulate in the Golgi of EPA_treated cells. Type IV particles (FIG. 5D, middle panel) have a similar size (FIG. 4) to small LpB particles (<25 nm; Shelness and Sellers, 2001, Curr. Opin. Lipidol. 12: 151-157). Type V particles (FIG. 5D, bottom panel), which represent the most numerous particle type in the Golgi had sizes (25-75 nm; FIG. 4) corresponding to those of apoB-reactive VLDL particles in the secretory pathway of rat liver (Alexander et al., 1976. J. Cell Biol. 69:241-263) and of VLDL particles (d<1.006 g/ml) isolated from Golgi fractions of rat liver (Verkade et al., 1993, J. Biol. Chem. 268:24990-24996). Type V particles in EPA-treated cells on average were larger (54.5 nm) than lipoprotein particles detected in oleate-treated cells (40 nm; Tran et al., 2002, J. Biol. Chem. 277:31187-31200). Enlarged d<1.006 g/ml VLDL particles (46.1 nm) have also been isolated from the lumen of choline-deficient rat livers (Verkade et al., 1993, J. Biol. Chem. 268:24990-24996).

Impaired VLDL assembly is thus associated with generation of a significant number of particles (FIG. 4, Type V) with enlarged lipoprotein morphologies (FIG. 5D, bottom panel); it remains to be determined whether these contain a full complement of apolipoproteins, including apoB100. If and how the aggregated apoB100 species associate with these particles also remains to be elucidated.

Unlike in oleate-treated cells where the majority of electron-dense particles were membrane-associated in cis-Golgi and luminal in trans-Golgi (Tran et al., 2002, J. Biol. Chem. 277:31187-31200), in EPA_treated cells four out of five identified particle types (Types I, II, III and V) retained significant membrane-association throughout the Golgi (Table II). Only the smaller Type IV particles were primarily membrane-associated in the cis-Golgi and luminal in trans-Golgi (FIG. 5A; Table II). Thus, the TEM data combined with the finding that apoB100 accumulates in the distal Golgi membrane (FIG. 3A, bottom panel), suggests increased membrane-association both for apoB100 and for larger lipoprotein-sized particles detected in the trans-Golgi.

Table II summarizes the percentage membrane associate of particles in the Golgi of EPA-treated cells.

TABLE II
Percent membrane association^{a} of particles
in the Golgi of EPA-treated cells

| | Golgi saccules | | | | TGN and | |
|---|---|---|---|---|---|---|
| | 1 | 2 | 3 | 4-6 | secretory | GAV |
| Type I | 100 | 84 | 80 | 73 | 56 | 33 |
| | (n = 8) | (n = 19) | (n = 15) | (n = 22) | (n = 9) | (n = 27) |
| Type II | 100 | 83 | 75 | | | 25 |
| | (n = 10) | (n = 6) | (n = 8) | (n = 0) | (n = 0) | (n = 4) |
| Type III | 100 | 83 | 83 | 82 | 73 | 46 |
| | (n = 5) | (n = 12) | (n = 23) | (n = 28) | (n = 15) | (n = 24) |
| Type IV | | | (1-3)^{a} | | | |
| | | | 67 | 50 | 20 | 45 |
| | | | (n = 18) | (n = 10) | (n = 5) | (n = 11) |
| Type V | 96 | 83 | 66 | 64 | 66 | 42 |
| | (n = 25) | (n = 94) | (n = 94) | (n = 107) | (n = 95) | (n = 155) |

^{a}Membrane association defined as the particle being either directly apposed to the Golgi limiting membrane or attached to it via a “membranous tab.”
^{b}Type IV particle diameters measured in combined Golgi saccules 1-3.

### Example 6—Sequstration of Lipid/Lipoprotein Particles into trans-Golgi Associated Vacuoles

Two Golgi stacks (GA1, GA2) consisted of four and five saccules [labelled 1 through 4 or 5), respectively (panel A). Saccule 1 is closely associated with the overlaying ER and has characteristic perforations (arrowhead) and thus represents the cis-end of the Golgi. Electron-dense particles (short black arrows) are present in the Golgi apparatus plus TGN and secretory vesicles (SV). Similar particles (long black arrows) are seen in GAV (small black asterisks) that are encased by cisternal membranes (dotted lines). The cisternal membranes are in continuum with ribosome (black arrowheads)-associated ER. Large vacuoles (large black asterisks) located further away from the Golgi have a dense, degradative content. Scale bar, 1 μm.

Panel B shows encasement of GAV (black asterisk) near the trans-end of Golgi (GA) by cisternal membranes (dotted lines). Spherical particles (black arrows) are present in the GAV. Buds (white arrows) and an invagination that contains several small vesicles and tubules (white asterisks) are associated with one of the GAV. Black arrowheads denote a microtubule. Scale bar, 0.4 μm.

The top of panel C shows close association and apparent fusion (white arrowheads) between a GAV (small black asterisk) containing electron-dense particles and a dense, degradative vacuole (large black asterisk) that lacks these particles. The middle of the image shows a GAV (small black asterisk) containing electron-dense particles (arrows) and having vesicles/tubulels (small white asterisks) in an invagination.

Panel D shows a 3D-model of two Golgi stacks (cis-most Golgi saccule, yellow; saccules 2-5, grey; TGN/SV, orange) and a group of GAV (medium blue) between them. Several of these vacuoles show invaginations in their limiting membranes, which accommodate small vesicles/tubules (royal blue). Homotypic fusion between adjacent particle-containing GAV (unlike the heterotypic fusion in panel C) is indicated with paired opposing arrowheads. Dilations (light blue) containing electron-dense particles are in continuum with trans-Golgi saccules; this continuity is evident within the section (double arrows) for the two dilations closest to the viewer. Perforations in cis-saccule are indicated by white arrowheads.

Panel E shows the lower Golgi stack from panel D rotated 180° along the x-axis and modeled to include cisternal membranes (red). Particle-filled GAV (medium blue) which did not obscure the trans-Golgi were included in the model. Two GAV (*1, *2) seen in equatorial view are associated with cisternal membranes along their periphery. The other two GAV (*3, *4) seen in “pole view”, are encased by cisternal membranes. The cisternal membranes (red) also encase (white stippled lines) lipid/lipoprotein containing dilations (light blue) that are in direct continuum with trans-Golgi saccules (double arrows indicate a continuity apparent within a section; single arrow indicate likely continuity between sections).

It has been reported previously that in EPA-treated cells, large and at least partially assembled lipoproteins were selectively targeted for degradation in a post-ER compartment by a mechanism that was sensitive to inhibition of PI 3-kinase (Fisher et al., 2001, J. Biol. Chem. 276: 27855-27863). Pulse-chase studies (FIG. 3) pointed to an event in or downstream of the distal Golgi in EPA_treated cells that may additionally explain the increased intracellular accumulation of apoB100 after 1-h (FIG. 2A), and also the lack of recovery of this accumulated apoB100 in the medium during 3-h chase (FIG. 2B). If as the data suggested, degradation of apoB100 occurred slowly, it was postulated that apoB100-containing lipoprotein assembly precursors and/or products may be detectable by TEM in an intracellular degradative compartment.

Notably, all five types of electron-dense particles (Type I-V), identified in the secretory compartments (Golgi, TGN, and secretory vesicles; FIG. 5A,B) were also identified in a population of GAV (FIG. 5C).

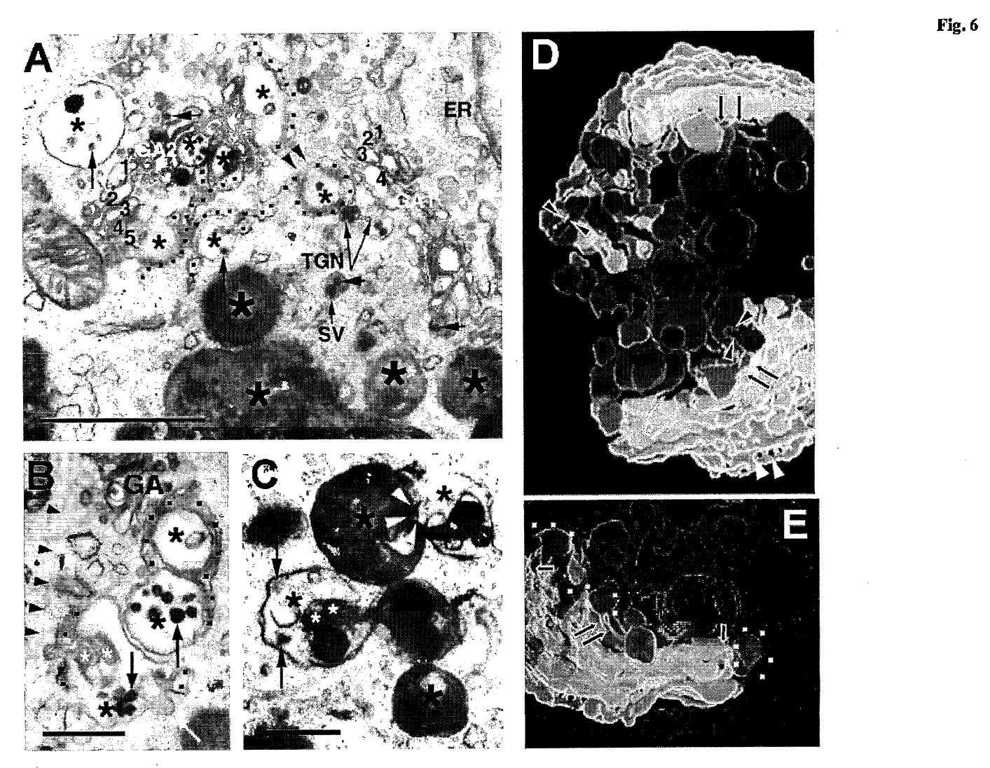

Unlike secretory vesicles, the GAV were encased by cisternal membranes (dotted lines, FIG. 6A) that showed continuity with ribosome-attached ER (FIG. 6A, arrowheads). The configuration of the cisternal membranes resembled that of “isolation membranes” formed during the early phase of autophagy (Mizushima et al., 2001, J. Cell Biol. 152:657-668). Autophagy is a PI 3-kinase-dependent process by which cells deliver cytoplasmic proteins and organelles to lysosomes or vacuoles for degradation through the formation of autophagosomes (Mizushima et al., 2001, J. Cell Biol. 152:657-6680). During autophagosome formation, a double-membraned isolation membrane, derived from the ER, TGN or de novo synthesized “phagophore” membranes, sequesters and enwraps target membranes or molecules. Closure of the isolation membrane leads to formation of an autophagosome. The GAV observed in this study had buds (FIG. 6B, white arrows) and/or invaginations that accommodated small vesicles/tubules (FIGS. 6B, C, white asterisks), suggestive of the fusion which occurs with late endosomes and/or lysosomes during conversion of autophagosomes to autophagolysosomes (Mizushima et al., 2001, J. Cell Biol. 152:657-668). The detection of apparent fusion profiles (FIG. 6C) between GAV and large, degradative vacuoles located near the Golgi region (FIGS. 6A, C) linked GAV to a degradative pathway, and was compatible with autophagy since heterotypic fusions occurs (Reggiori and Klionsky 2002, Eukaryot. Cell 1:11-21).

The extent of the GAV-compartment and its' relationship to the Golgi apparatus and secretory vesicles was further revealed in a 3D serial section model (FIGS. 6D, 6E). In this model, and in our library of serial sections, particle-filled dilations of trans-Golgi saccules (FIG. 6E, light blue) were encased by cisternal membranes (white dotted lines) that were continuous with membranes (red) that enveloped GAV (*3-4). This raises the possibility that particle-filled GAV originate from the trans-Golgi.

Next, to confirm that GAV function in lipoprotein metabolism, the particle content of GAV was compared to that of the Golgi and TGN. The relative occurrence of the five types of particles in the GAV

(I:II:III:IV:V=12%:2%:11%:5%:70%; n=221) was nearly identical to that in trans-Golgi saccules 4-6+TGN/secretory vesicles

(I:II:III:IV:V=12%:4%:13%:5%:66%; n=284), suggesting that sorting of specific particle types into the GAV does not occur. Sequestration of all particle-types into the GAV confirms that this organelle serves a role in lipoprotein metabolism. The similar particle-content in Golgi versus GAV is in accord with autophagic degradation typically being a “bulk” degradative compartment (Mizushima et al., 2003, Int. J. Biochem. Cell Biol. 35: 553-561). However, particles sequestered into the GAV exhibited altered particle-particle associations relative to those in the secretory pathway. While in the Golgi, particles were detected either singly or in a paired arrangement (one Type I, II or III particle and one Type IV or V particle, FIG. 5B), particles in the GAV more frequently were clustered (one Type I, II or III particle surrounded by multiple Type IV or V particles; FIGS. 5C, E).

In addition, particles in the GAV showed significant alterations in membrane association relative to in the secretory pathway. Type I-III and Type V were all less membrane-associated in the GAV than in the Golgi, TGN/SV (Table II). While the significance of the altered particle-particle and particle-membrane associations is unclear, these findings help to confirm that the GAV comprise a cellular compartment distinct from secretory compartments.

TEM thus identified a compartment of GAV, which by several morphological criteria (peripheral association with ER, fusion profiles with advanced degradative vacuoles, “bulk” sequestered content) resemble autophagosomes, and which sequester lipoprotein/lipid type particles.

### Example 7—Immunofluorescent Localization of apoB and Map1LC3

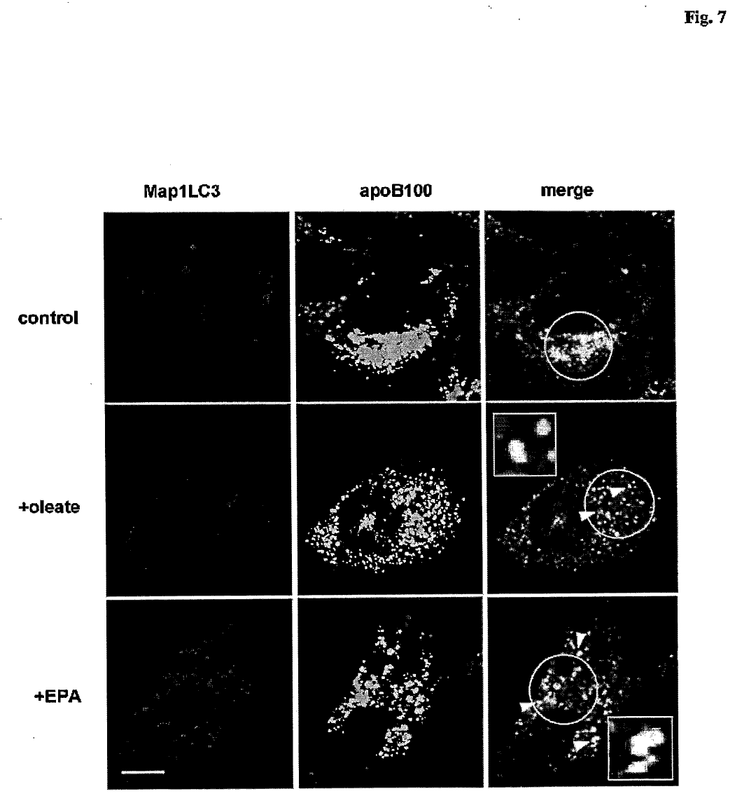

Cells pretreated with none (control), oleate or EPA were permeabilized and blotted with anti-human apoB antibody (apoB) and anti-rat Map1LC3 antibody (Map1LC3), respectively. The secondary antibody for apoB was conjugated with Alexa Fluor™488 (green), and that for Map1LC3 was conjugated with Alexa Fluor™594 (red). The circles in the merge images of FIG. 7, show redistribution of Map1LC3 into the apoB-rich region in oleate- or EPA-treated cells. The arrowheads of FIG. 7 show co-localization of Map1LC3 and apoB (magnified in insets). The scale bar for FIG. 7 is 10 μm.

To confirm that GAVs correspond to autophagosomes and sequester lipoprotein assembly precursors and/or products, indirect double immunofluorescence studies were carried out to establish possible co-localization between apoB100 and Map1LC3. A group of ATG (autophagy) gene products are required during autophagosome formation, including Map1LC3 that is recruited from the cytosol to the isolation membrane via a PI 3-kinase-dependent process (Mizushima et al., 2001, J. Cell Biol., 152: 657-668; Kabeya et al., 2000, EMBO J. 19:5720-5728). In comparison to controls, both EPA and oleate treatment induced autophagy, as shown by enhanced penetration of Map1LC3 staining into the apoB100-rich perinuclear area with partial co-localization of Map1LC3 and apoB100 (FIG. 7). Co-localization of apoB100 and Map1LC3 was more pronounced in EPA—than in oleate-treated cells.

### Example 8—EPA Treatment Enhances Autophagy

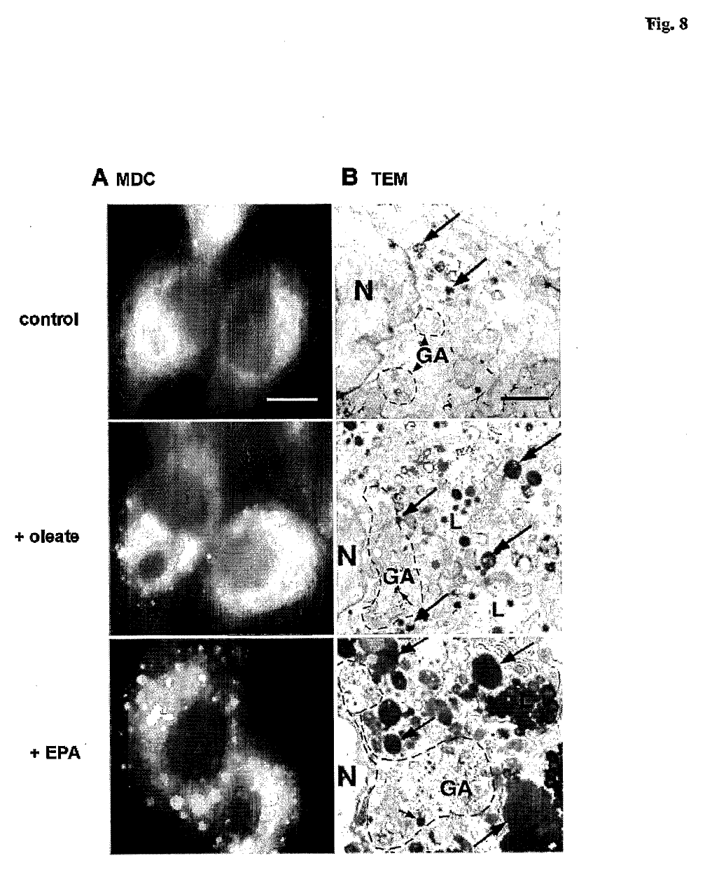

FIG. 8A, illustrates monodansylcadaverine (MDC)-labelling of control, oleate- and EPA-treated cells. The scale bar is 10 μm.

FIG. 8B, are TEM images of control, oleate- and EPA-treated cells. The large arrows denote dense vacuoles near the Golgi apparatus (GA; stippled). The small arrows denote small dense vacuoles within the Golgi region of oleate- or EPA-treated cells. (N) refers to the nucleus and (L) refers to lipid droplets.

In cells treated with the same dose of EPA or oleate (0.4 mM), formation of autophagolysosomes was also more prominent in EPA—than in oleate-treated cells, as demonstrated by the enlargement of dense vacuoles reactive with MDC, a specific marker of autophagolysosomes (Biederbick et al., 1995, Eur. J. Cell Bio. 66: 3-14) (FIG. 8A). The size and distribution of MDC-reactive vacuoles in EPA-treated cells resembled that of dense, degradative vacuoles located outside the Golgi region as visualized by TEM (FIG. 8B, arrows). The TEM and immunocytochemistry data together suggest that EPA treatment enhanced autophagy, and that a proportion of lipid and lipoprotein particles were diverted from the secretory pathway into an autophagic degradative compartment.

### Example 9—18:1(n-9) TG is Utilized for VLDL Assembly and Secretion

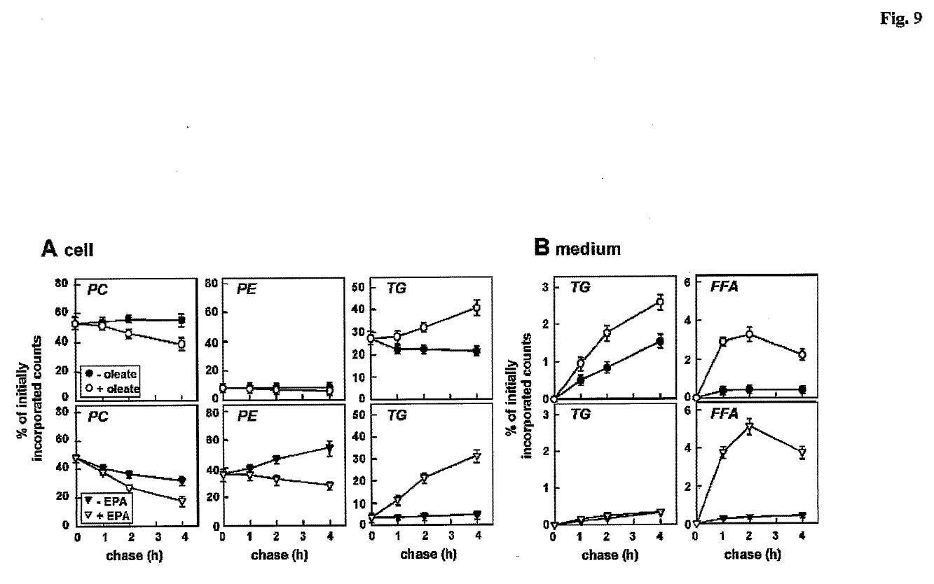

Cells were labelled with [¹⁴C]oleate for 2 h, and chased in the presence or absence of 0.4 mM exogenous oleate for 1, 2 and 4 h (FIGS. 9A & 9B, top panels). Similarly, cells were labelled with [³H]EPA for 2 h and chased in the presence or absence of 0.4 mM EPA for up to 4 h (FIGS. 9A & 9B, bottom panels). At each chase time, total lipids were extracted from the cells (FIG. 9A) and medium (FIG. 9B), respectively, resolved by TLC, and radioactivity associated with PC, PE, TG and free fatty acid (FFA) was quantified by scintillation counting. Data are expressed as percent of total radioactivity incorporated at the end of 2-h labelling (2.4×10⁶ in [¹⁴C]oleate-labelled cells and 6.1×10⁵ cpm in [³H]EPA-labelled cells. The results are the averages of two independent experiments with error bars showing the range of deviations.

The inventors' previous work suggested that TG synthesized via phospholipid remodelling is utilized during the second-step VLDL assembly (Tran et al., 2002, J. Biol. Chem. 277: 31187-31200). To gain an insight into the mechanism by which EPA treatment impairs VLDL assembly, we compared TG synthesis via phospholipid remodelling between oleate- and EPA-treated cells. The cells were labelled with [¹⁴C]oleate or [³H]EPA for 2 h, and chased up to 4 h in the presence of unlabeled exogenous oleate or EPA, respectively. At the end of 2-h labelling (i.e. at 0 h of chase), PC, PE and TG accounted for 53%, 8%, and 27%, respectively, of total [¹⁴C]-labelled cellular lipids in [¹⁴C]oleate-treated cells (FIG. 9A, top panels), and 48%, 36%, and 3%, respectively, of total [³H]-labelled cellular lipids in [³H]EPA-treated cells (FIG. 9A, bottom panels). Thus, [¹⁴C]oleate was mainly incorporated into PC and TG, whereas [³H]EPA was incorporated into PC and PE but not TG. During chase, the counts of [¹⁴C]oleate-labelled PC and PE were relatively constant in the absence of exogenous oleate (FIG. 9A, closed circles in top panels), which, in accord with previous observations (Tran et al., 2000, J. Biol. Chem. 275:25023-25030), indicates a low rate of phospholipid turnover under basal conditions (i.e. no exogenous oleate). In contrast, exogenous oleate treatment stimulated the turnover of [¹⁴C]oleate-labelled PC and the transfer of 18:1 (n-9) acyl chain into TG (FIG. 9A, open circles in top panels).

In [³H]EPA-labelled cells, the counts associated with PC decreased with a concomitant increase in PE during chase (FIG. 9A, closed triangles in bottom panels), which, as shown previously (Balsinde 2002, Biochem. J. 364:695-702), indicates that transfer of 20:5 (n-3) acyl chains from PC to PE occurred under basal conditions (i.e. no exogenous EPA). Addition of exogenous EPA into the chase medium stimulated turnover of both [³H]EPA-labelled PC and PE, and the 20:5 (n-3) acyl chain derived from PC and PE was transferred into TG that accounted for ˜30% of total [³H]EPA radioactivity in the cells at the end of 4-h chase (FIG. 9A, open triangles in bottom panels). Remodelling of phospholipid induced by exogenous fatty acids was also evident by the release of free [¹⁴C]oleate or [³H]EPA into the medium (FIG. 9B, open circles & triangles in right panels). Thus, during phospholipid remodelling, both 18:1(n-9) and 20:5(n-3) acyl chains derived from deacylation of the respective phospholipids are utilized for TG synthesis. A striking difference was observed between the secretion of [¹⁴C]oleate-TG or [³H]EPA-TG during chase. The [¹⁴C]oleate-TG was secreted and its secretion was further stimulated by exogenous oleate, whereas [³H]EPA-TG was not secreted regardless of whether exogenous EPA was present (FIG. 9B, left panels). These results suggest that while 18:1(n-9)-TG was utilized for VLDL assembly and secretion, the 20:5(n-3)-TG was not.

### Example 10—TG Synthesized Via PE Remodelling is Preferentially Shunted to Cytosol

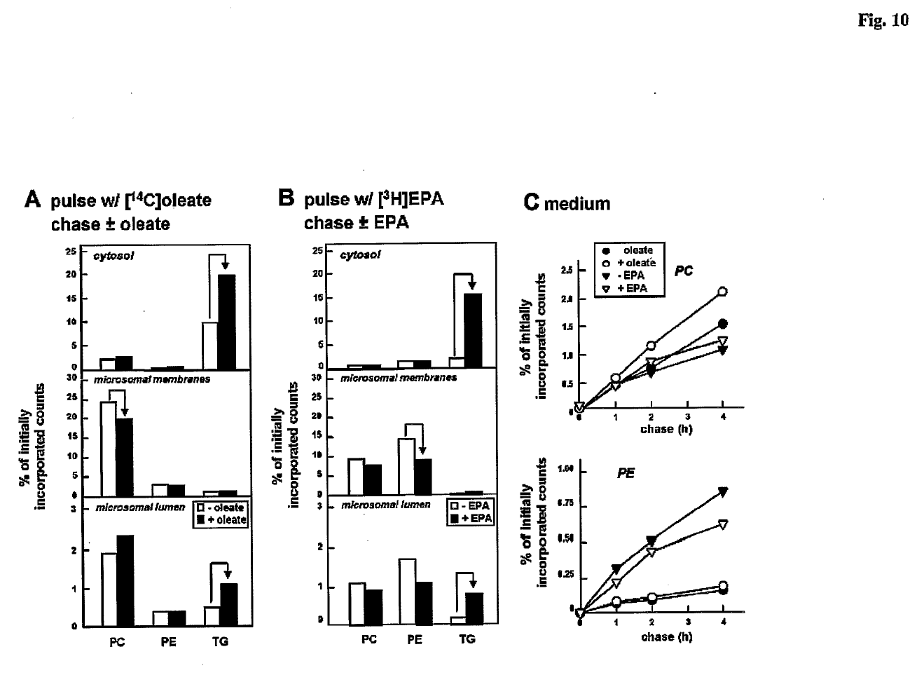

Cells were labelled with [¹⁴C]oleate for 2 h, and chased in the absence (FIG. 10A, open bars) or presence (FIG. 10A, closed bars) of 0.4 mM oleate for 4 h. Cells were labelled with [³H]EPA for 2 h, and chased in the absence (FIG. 10 B, open bars) or presence (FIG. 10B, closed bars) of 0.4 mM EPA for 4 h. The cells were homogenized and the intracellular compartments (i.e. cytosol, microsomal membranes and microsomal lumen) were fractionated, Lipids were extracted from each fraction and resolved by TLC, and quantified by scintillation counting. Data are averages of duplicates and expressed as percent of total radioactivity incorporated at the end of 2-h labelling. The range of deviations (not shown) was less than 5% from the average values.

The differential utilization of 18:1(n-9)-TG and 20:5(n-3)-TG for VLDL secretion between oleate- and EPA-treated cells may reflect different compartmentalization of 18:1(n-9)-TG and 20:5(n-3)-TG accessible for VLDL assembly. It was hypothesized that the asymmetric distribution of PC and PE on the microsomal membranes (i.e. PC enriched on the lumenal side and PE on the cytosolic side), together with the changes in PC-to-PE ratio upon EPA and oleate treatment, might result in TG partitioning into different pools (e.g. cytosolic pool for storage and microsomal pool for VLDL assembly).

To test this hypothesis, the intracellular distribution of radio-labelled lipids was contrasted between two groups of cells that had been respectively pulse-labelled with [¹⁴C]oleate- or [³H]EPA, and chased with media±oleate (FIG. 10A) or EPA (FIG. 10B). At the end of chase, the majority of [¹⁴C]oleate was associated with PC whereas the majority of [³H]EPA was associated with PE and PC in microsomal membranes (FIG. 10A, B, middle two panels). Addition of exogenous oleate or EPA during chase caused a decrease of [¹⁴C]oleate or [³H]EPA associated with the membrane phospholipids and a concomitant increase of [¹⁴C]oleate or [³H]EPA associated with cytosolic TG (top panels). The magnitude of increase in cytosolic TG was much greater for 20:5(n-3)-TG than that for 18:1(n-9)-TG. In a separate experiment where cells were pulse-labelled with [³H]glycerol and then chased in the presence of either oleate or EPA, the increase in cytosolic [³H]glycerol-TG was also more pronounced in EPA-treated cells than in oleate-treated cells (data not shown). Thus TG synthesized via [³H]EPA-labelled PE remodelling was preferentially shunted to cytosol. However, both [¹⁴C]oleate-labelled TG and [³H]EPA-labelled TG showed increases in the microsomal lumen during chase (bottom panels) which along with the enhanced detection of lipid-type droplets in the Golgi by TEM, indicates that the impaired VLDL assembly in EPA-treated cells is not simply a consequence of TG being unavailable at the VLDL assembly site.

### Example 11—PC and PE Content in Membranes of Subcellular Organelles in Oleate and EPA Treated Cells

Table III summarizes the PC and PE content in membranes of subcellular organelles in oleate and EPA treated cells.

TABLE III
PC and PE contents in membranes of subcellular organelles in oleate- or EPA-treated cells

| | Control | | Oleate | | EPA | |
|---|---|---|---|---|---|---|
| | PC | PE | PC | PE | PC | PE |
| | Peak area × 10⁻⁷ (% of control)^{a} | | | | | |
| Distal | 25.2 [24.1; 26.3] | 1.6 [1.4; 1.8] | 43.6 [42.5; 44.6] | 1.7 [1.5; 1.9] | 23.6 [23.2; 24.0] | 4.3 [3.3; 5.3] |
| | (100) | (100) | (173) | (106) | (94)* | (270)* |
| cis/medial | 57.8 [48.1; 67.6] | 3.3 [3.2; 3.4] | 62.1 [57.0; 67.1] | 3.7 [3.6; 3.7] | 39.8 [33.9; 45.8] | 4.2 [3.6; 4.8] |
| | (100) | (100) | (107) | (112) | (70)* | (127) |
| ER | 89.8 [81.5; 98.1] | 2.5 [2.1; 3.0] | 164 [136; 192] | 4.3 [4.0; 4.6] | 96.5 [90.1; 103] | 5.4 [4.8; 6.0] |
| | (100) | (100) | (182) | (172) | (108)* | (216) |
| Total | 173 [154; 192] | 7.5 [6.7; 8.2] | 270 [236; 304] | 9.7 [9.1; 10.2] | 160 [147; 173] | 13.9 [11.7; 16.1] |
| | (100) | (100) | (156) | (129) | (92)* | (185)* |

^{a}Lipids extracted from membranes of distal Golgi, cis/medial Golgi and ER were subjected to tandem mass spectrometry to quantify PC or PE mass. The data are means of two independent experiments whose values are shown in squared brackets. The percent change PC and PE in oleate- or EPA-treated cells over the corresponding value in control cells (set as 100) is shown in parentheses.
*The changes marked with asterisks indicate marked reduction or increase in PC and PE between EPA- and oleate-treated cells.

It has been shown that in yeast, lipidation of Apg8/Aut7 (a Map1LC3 orthologue) by PE is essential for the initial assembly of autophagocytic membranes (Mizushima et al., 2001, J. Cell Biol., 152: 657-668). The effect of EPA and oleate treatment on the content and composition of PC and PE associated with intracellular membranes was determined using tandem mass spectrometry. Total PE mass was increased by 85% in EPA-treated cells, with a 170% and 116% increase occurring in the distal Golgi and ER, respectively (Table III). Total PC mass was unaffected by EPA treatment as compared with untreated control, but was lower than that of oleate-treated cells.

There was a moderate increase in total PE mass (by 29%) with oleate treatment which occurred primarily in the ER (by 72%). Total PC mass associated with intracellular microsomes was increased by 56% by oleate treatment; most of the increase occurred in the ER (by 82%) and distal Golgi (by 73%) (Table III). Thus, EPA caused a massive increase in PE content.

1-30. (canceled)

31. A method of reducing serum levels of triglycerides or VLDL, the method comprising administering a therapeutically effective amount of an autophagocytosis inducing compound to a patient in need thereof.

32. The method of claim 31, wherein the autophagocytosis inducing compound is selected from the group consisting of Map1LC3, GABARAP, GATE16, and Class III PI3 kinase.

33. Use of an autophagocytosis inducing compound for preparing a medicament useful for reducing serum levels of triglycerides or cholesterol.

34. The use of claim 33, wherein the autophagocytosis inducing compound is selected from the group consisting of Map1LC3, GABARAP, GATE16, and Class III PI3 kinase.

35. A method of treating or preventing a disorder in a patient in need of such treatment or prevention, the method comprising administering a therapeutically effective amount of an autophagocytosis inducing compound, wherein the disorder is selected from the group consisting of hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia, or a combination thereof.

36. The method of claim 35, wherein the autophagocytosis inducing compound is selected from the group consisting of Map1LC3, GABARAP, GATE16, and Class III PI3 kinase.

37. Use of an autophagocytosis inducing compound for the preparation of a medicament useful for treating or preventing a disorder selected from the group consisting of hypertriglyceridemia, hyperlipidemia, hypercholesterolemia, hyperlipoproteinemia, hypertriglyceridemia, hyperlipidemia, hypercholesterolemia, hyperlipoproteinemia, atherosclerosis, arteriosclerosis, peripheral artery disease, coronary artery disease, congestive heart failure, myocardial ischemia, myocardial infarction, ischemic stroke, hemorrhagic stroke, restinosis, diabetes, insulin resistance, metabolic syndrome, renal disease, hemodialysis, glycogen storage disease type I, polycystic ovary syndrome, secondary hypertriglyceridemia, or a combination thereof.

38. The use of claim 37, wherein the wherein the autophagocytosis inducing compound is selected from the group consisting of Map1LC3, GABARAP, GATE16, and Class III PI3 kinase.

39. A method of identifying autophagocystosis modulating compounds, the method comprising:
(a) providing a control cell culture system and a test cell culture system;
(b) administering a test compound to cells in the test cell culture system; and
(c) assaying for an autophagocytosis marker in the control cell culture system and the test cell culture system, wherein an abnormal value for the autophagocytosis marker in the test cell culture system as compared to the control cell culture system indicates that the test compound modulates autophagocytosis.

40. The method of claim 39, wherein the autophagocytosis marker is a VLDL or a VLDL precursor in an ER or a Golgi cell fraction.

41. The method of claim 40, wherein the VLDL precursor is a PC or a PE moiety containing lipid.

42. The method of claim 41, wherein the PC moiety containing lipid is 18:1 (n-9) PC, wherein the PE moiety containing lipid is 20:5(n-3) PE.

43. The method of claim 39, wherein c) assaying comprises detecting degree of co-localization of apoB100 and Map1LC3 by immunofluorescence.

44. A method of identifying autophagocystosis inducing compounds, the method comprising:
(a) providing a control cell culture system and a test cell culture system;
(b) administering a test compound to cells in the test cell culture system; and
(c) assaying for an autophagocytosis marker in the control cell culture system and the test cell culture system, wherein an abnormal value for the autophagocytosis markers in the test cell culture system as compared to the control cell culture system indicates that the test compound modulates autophagocytosis.

45. The method of claim 44, wherein the autophagocytosis marker is a PC or a PE moiety containing lipid in a ER or a Golgi cell fraction.

46. The method of claim 45, wherein the PC moiety containing lipid is 18:1(n-9) PC, wherein the PE moiety containing lipid is 20:5(n-3) PE.

47. The method of claim 44, wherein c) assaying comprises detecting degree of co-localization of an apoB1 protein and a Map1LC3 protein by immunofluorescence.

48. The method of claim 39, wherein the cells are hepatocytes or hepatoma cells.

49. The method of claim 48, wherein the hepatocytes are rat hepatocytes which express a human apoB100 protein.

50. The method of claim 48, wherein the hepatoma cells are rat hepatoma cells which express a human apoB100 protein.

51. The method of claim 50, wherein the rat hepatoma cells are McA-RH-7777 cells.

52. The method of claim 49, wherein the human apoB100 protein is fused with a tag.

53. The method of claim 52, wherein the tag is a fluorescent protein.

54. The method of claim 52, wherein the tag is tetra-cysteine having the sequence Cys-Cys-X-X-Cys-Cys, wherein X is any amino acid.
